(19) United States
(12) Patent Application Publication
(10) Pub. No.: US 2023/0140306 A1
(43) Pub. Date: May 4, 2023

(54) HORIZONTAL CELLS

(71) Applicant: UCL BUSINESS LTD, London (GB)

(72) Inventors: Matteo RIZZI BRIGNOLI, London (GB); Kate Louise OVERSBY-POWELL, London (GB)

(21) Appl. No.: 17/798,799

(22) PCT Filed: Feb. 12, 2021
(86) PCT No.: PCT/GB2021/050358

§ 371 Date: Aug. 10, 2022

(30) Foreign Application Priority Data
Feb. 14, 2020 (GB) 2002073.1

(51) Int. Cl. A61K 48/00; A61P 27/02

(52) U.S. Cl. CPC A61K 48/005; A61P 27/02

(57) ABSTRACT

The present invention relates to methods of improving vision. Specifically, the invention relates to the use of a nucleic acid molecule to activate (hyperpolarize) horizontal cells in the mesopic and photopic illumination ranges and the use of said nucleic acid molecule to improve visual function, for example following degeneration or loss of the ‘macula’.

### FIELD OF THE INVENTION

The present invention relates to methods of improving vision. Specifically, the invention relates to the use of a nucleic acid molecule to activate (hyperpolarize) horizontal cells in the mesopic and photopic illumination ranges and the use of said nucleic acid molecule to improve visual function, for example following degeneration or loss of the ‘macula’.

### BACKGROUND OF THE INVENTION

The retina detects light, processes and transmits visual information to the brain. Photons hitting a photoreceptor are transduced into an electrical signal and fed into parallel bipolar cell (BC) pathways that extract different visual features and convey this information to the retinal ganglion cells, the output neurons of the retina (47). The signals along this ‘vertical’ pathway—from photoreceptors to BCs to retinal ganglion cells—are modulated by inhibitory neurons, including horizontal cells (HCs). Horizontal cells are laterally-projecting interneurons that provide feedback and feedforward signals to photoreceptors and bipolar cells respectively.

Center-surround organization is a fundamental feature of the visual system. It has been observed in the retina and in the brain, both at single-cell and perceptual level. According to the standard hypothesis of center-surround organization, competitive antagonism between surround and center enhances contrast differences in adjacent parts of a field of view. This appears to be exemplified by the “simultaneous contrast illusion” whereby a difference in luminance is perceived when a central grey area is surrounded by a dark versus a light surround. Schematics typically indicate this opposition using ‘plus’ and ‘minus’ signs to demarcate the different effect on the response.

The hypothesis of a competitive surround has never been challenged, but its intuitive appeal has made it a textbook axiom in neuroscience. The data presented herein challenges the idea that center-surround organization is involved in ‘lateral contrast enhancement’.

### SUMMARY OF THE INVENTION

By using a combination of psychophysics in subjects with normal vision and with specific inherited retinal diseases, together with retinal circuit manipulation in equivalent mouse models, the inventors have demonstrated that the current model of center-surround organization in the retina needs to be re-examined.

The inventors identify for the first time that center-surround organization in the retina does not simply act in an antagonistic (competitive) manner. Instead, the inventors have shown that mean luminance in the surround of a stimulus tethers contrast encoding in the ‘center’. As a result, contrast changes in time or space are never transmitted to downstream neurons in absolute terms, but always relative to the surrounding context. Thus the center-surround organization is fundamentally adaptive in nature, and adjusts peak contrast sensitivity to its own mean luminance. The fast lateral adaptation provided by these circuits drives the differential effects of luminance and contrast in the surround.

In light of this data, the inventors have reinterpreted several observations and phenomena, including the classic receptive field work by Kuffler (3), ‘surround contrast suppression’(19) and contrast enhancement (4). This reinterpretation leads to a different understanding of a fundamental feature of perception.

Importantly, the inventors have identified a specific circuit (cone-horizontal cell-rod) that mediates this interaction in mouse.

The present inventors have shown that, in a healthy retina, the red and green cone photoreceptors signal the light level they are detecting to the rod photoreceptors and cone photoreceptors in the surrounding retina. In particular, cone photoreceptor cells hyperpolarize horizontal cells, which then enable the operating range of targeted rod and cone photoreceptors to be adjusted. As a result, the optimum sensitivity range of the postsynaptic photoreceptors re-tunes to a light level equivalent to the brightness of the surrounding area. It is shown that under these circumstances, the rod photoreceptors can make a positive contribution to vision even under mesopic light conditions.

In mice, only one type of horizontal cell (a cone to rod horizontal cell) is known to exist. In these models degeneration of cone photoreceptors deprives horizontal cells of their natural input. In the absence of the feedback mechanism, the rod photoreceptors cannot contribute (or cannot contribute as effectively) to overall vision under brighter light. The feedback mechanism from cone photoreceptor to rod photoreceptor functions through horizontal cells, which form lateral connections between the synapses that connect the photoreceptors to the bipolar cells. The inventors show that in mice lacking cone photoreceptor function, artificially hyperpolarising horizontal cells can shift the optimum light sensitivity range of the rod photoreceptors in a manner similar to the feedback by the cone photoreceptors. As a result, vision in the mice improved in brighter light. Thus, the data presented herein show manipulation of the lateral adaptive mechanisms may be used to make rod photoreceptors more cone-like at mesopic light levels.

A similar beneficial effect is expected in patients. However in humans more than one type of horizontal cell is known to exist. The inventors have shown that activation of cones in the surround is able to improve cone mediated vision, suggesting that lateral luminance adaptation is also important for proper function of cone mediated vision. Accordingly, therapies that target horizontal cells in patients with local loss of cones (e.g., macular degeneration) could experience an improvement of remaining cone as well as rod vision. There is the potential to differentially target cone and rod horizontal cell types. Thus, the inventors have identified a previously unknown function of the classic center-surround organization in the retina.

These results are surprising. One skilled in the art would not consider improving the function of rod photoreceptor cells by lateral input. Rod photoreceptors are generally considered to be unable to function at high light intensities because they “saturate” their range of operation due to the strong amplification required for their sensitivity. Saturation of rod photoreceptor signalling is potentially disruptive to visual processing, because ultimately rod and cone generated signals have to be integrated within the retina. To avoid this interference at mesopic light levels, cone activity is thought to suppress rod activity.

This organization may be exploited to improve vision in patients with retinal degeneration. Loss of lateral input in common retinal degenerative disorders, such as macular degeneration, is shown to compromise the remaining vision. Horizontal cells are thus identified as a potential therapeutic target. The horizontal cell is a good clinical target for a number of reasons. Firstly, it is known that when photoreceptors degenerate and are lost from the retina most of the other retinal cell types remain and retain their connections. This would allow the targeting of horizontal cells inside the lesion, meaning that the treatment would not have to disrupt and potentially compromise remaining vision (for example, on-target effects are unlikely to directly cause visual side effects). Secondly, horizontal cells are a laterally projecting cell type, and thus direct treatment of a small area is likely to create a treatment effect over a much larger area. The invention is therefore highly relevant for retinal dystrophies.

The described effect can be global, for conditions where all cone photoreceptors are affected, or local if there is an area of missing or dysfunctional cone cells. The vast majority of cone photoreceptors are localized in a small area at the center of the retina, the macula. The macula is the most important region of the retina for accurate vision. Patients with a degeneration of the macula generally use the surviving retina just outside the lesion for fixation and high visual acuity tasks. However, this area has far fewer cone photoreceptors and the rod photoreceptors that are present are affected by the lack of feedback from the cone photoreceptors in the degenerated retina.

For conditions lacking cone function, the data suggest that a lack of gain control via horizontal cells would lead to reduced contrast sensitivity. This effect is observed both in patients and mice with achromatopsia. Lack of lateral gain control may also explain why patients with achromatopsia describe daylight as ‘dazzling’ and are averse to light levels that are considerably lower than those tolerated by normal vision subjects. For conditions involving degeneration of cone cells, such as age-related macular degeneration (AMD) and Stargardt disease, the data presented herein reveal that gain control is impaired in areas adjacent to a degenerate part of the retina and that quality of vision is worsened as a result.

Improvement of visual function adjacent to degenerated macula regions could be greatly beneficial since visual acuity declines rapidly with increasing eccentricity. Importantly this would be true for patients in which foveal function is spared as well as those that rely on eccentric fixation. Furthermore, the data suggests that gene supplementation or cell replacement strategies aiming to preserve or restore photoreceptor function may sustain an improvement in vision over a considerably wider area than the one directly treated, due to preserved or restored lateral input.

The nucleic acids, pharmaceutical compositions and methods of the invention may be used for the treatment of disorders involving degeneration of photoreceptors. In particular, it is envisioned that the present invention may be used for the treatment of patients with advanced cone dystrophy or macular dystrophy (inherited MD such as Stargardt disease, as well as AMD). The invention could also benefit achromatopsia patients who show a pronounced loss of cone photoreceptor cells.

The present invention therefore provides an expression construct comprising a promoter operably linked to a gene product, wherein the expression of the gene product in horizontal cells improves vision. In a preferred embodiment, the invention provides an expression construct comprising a promoter operably linked to a gene product, wherein the expression of the gene product in horizontal cells improves mesopic and/or photopic vision.

Also provided is:
a vector or host cell comprising the expression construct of the invention;
a pharmaceutical composition comprising the expression construct or vector of the invention;
the vector or pharmaceutical composition of the invention, for use in a method of treating a retinal dystrophy; and
a method for treating a cone dystrophy or macula dystrophy, the method comprising administering the vector or pharmaceutical composition of the invention to a subject.

SEQUENCE LISTING

SEQ ID NO: 1-mouse Gja10 partial promoter sequence

| | |
|---|---|
| gcaatagaaa ccctaactaa aacaattcta tttaaaattt tcttcatata tatgtatgaa | 60 |
| aacaggtaag gtttacataa atgtttttat taaataaaat aatattttct tttataaaaa | 120 |
| acatttattt tatgtgtatg aatgttttgc ttggctgtgt ctgtgcacca cttccaggct | 180 |
| tggttcacac agtggtcaga agagagtgga tctcttgaga gtagagttat gaatggttgt | 240 |
| aatctactct gtgggtactg ggaaccaatt caatcataac caaaaccaaa gccatcttcc | 300 |
| agtcccagtc atattttctt agaaacccgt tgtccctttc ttttcttttt tacctgtttt | 360 |
| ctacctctcc tgcctcttct ttctcccttc taattcttcc catatccata ctcctcgact | 420 |
| tcctttccat gctcacacac acacacacaa aatgtatgtg tgtgtgtata catacatata | 480 |
| tatgtatgta tgtatgtatg tatatgtata cacacatata tacacacata catatataca | 540 |
| catgtataca tatataaaca cacatatata tatgtgatca tgtatatact catatatatg | 600 |
| cttatataat catatatctc ttatacattg tatatgtgta catttatgga attttatttt | 660 |
| attcagtgat taaaagtctt acaacaacct acagattaat ttatgtgagg aataatagtg | 720 |
| agtttccaat tccttttggt ttttcaggtg aaattacaat atatgtgtgt cccacacctc | 780 |
| atatatacca tgactatggt tgagtttgtg gcttctgaga taatgcctag gtagaggtaa | 840 |
| cacttcagaa aaatattgga gagatgaatt ttttaaaaga tttgtttgtt tgtttattta | 900 |
| tttatttatt tattacgcat aatgttctgc ctgcacttat gcctgcaagc caggagcggg | 960 |
| caccagacct cattacagat ggttgtgagc caccatgcag ttgctgggaa ttgaactcag | 1020 |
| gacctctgga agagtagaca gtgcttttaa cctctgagcc aactctccag ccctgaagag | 1080 |
| atgaatttaa aaattaaaaa ttatgccacg gttgttggtt tcaggatgga agctgagaaa | 1140 |
| actcatctca gagatagaag cttaaaaatg aaagctttgt tttctgagtg ctcagggagg | 1200 |
| tggccacttc agaatctgaa ctgcttccca gaaaggagat ggtacatttt taaaggatgg | 1260 |
| gagcacaaag caagtgggta gctgggagga gctgggggag ctgctgtgtt atccagcttt | 1320 |
| attttgacat tacaggttaa gctgggttaa gcaaggaagt accagttaga gactgggatg | 1380 |
| catccaggac acctggcttc aaggttctaa ttcagctttt gcagccaggt cccctcctgg | 1440 |
| actctggact ggaattttag aatgataagg gagcaaagaa gtgggcaagc agcacgtagt | 1500 |
| caattagggt ataacaggca attggatata tattttatag tttatgtacc ctggtttagg | 1560 |
| taatagcagt ttccatattt ttttactggt taacagatga ttaagaaaac attggagaag | 1620 |
| tgggacagaa gttctttcct aggtgtagga acttgaactt gtttgaccct gccagcaaga | 1680 |
| tggggaattt gtccttgaga tgggtggatt cagacaactg tctccataca atttgcccct | 1740 |
| gagatatctg gactcagaca actgtttaca ccagaggctg cttggctatg gggaagtacc | 1800 |
| cagctctcct acggcatggg accttgaatt tagtttcttc ttatggttaa atatagtctt | 1860 |
| aaagcaaaat agtagtactt agaataagtt tcttttgttt gttccttaca attgtctact | 1920 |
| tcaatttaat tacattttac cctatgattt tcatatctaa aaagccttcc acatattaga | 1980 |
| aatctttagg ttatcatgat ggctagtctt cacacaaact agagctatct gtggggaggg | 2040 |
| aaacttaatt gagaaaatgc ttccataaga tttggctgta aggtggtttt ttagtgattg | 2100 |
| atgtggggag ggcccagtgc attatggatg attccattcc aaggctggtg gttctaggtt | 2160 |
| ctagaaaaaa gcaggatgag caagccatga aaagcagacc agtaagcagc acccttccat | 2220 |
| agcctctgca tcagctcctg cctccaggtt ccagccctgc ttgagttcct gtcctgactt | 2280 |
| tttccaatga tgggttatga tctggaagta taagctgagt aagcaaaccc tttccttccc | 2340 |
| aacttgcttt ttggtcatgg tgttttcata gcagccatag aaaccctaag acagcgtttt | 2400 |
| gagtgttttg atctaattga accttgaaaa acatagtttt tccaggataa aaaacagtaa | 2460 |
| cttgtgtatg acacttatac cttgattatt tcctagactc caagctttct agagaaacca | 2520 |
| tttatactaa cttttccaag tttgcaagaa ttccataaag acacaagaca cggtggcaca | 2580 |
| gccctttaat tctagcactt gggagtcaga ggcaggcaac tctgagtttg agacgagtct | 2640 |
| gctatacaaa gtggattcca ggacagttag ggctgttatg cagagaaatt ctgtcttgaa | 2700 |
| aaaccaagaa aattttcaat gaagacctgg ctttccagat gtgttttagt ggcactgttt | 2760 |
| ttgtgactgc ctcaacatta ccactaactt gacctgaagc agctcccaag gatttgcctg | 2820 |
| gctgatttct ctttacaagt gcttcatctt acaatggtag cacgtgtggt atatgaggag | 2880 |
| atcctacttt gctttccctg ttgacagtta tttcatgaaa gactcaaggg aatatattga | 2940 |
| tatcatctgt gtagagagat ccagacacca tggagagact ctacttgcag ggggcgcacc | 3000 |
| ttttcaggaa atgagacaga aaatgaaaac accacaacag tcttctaggt taaatgctca | 3060 |
| atgagtggta gtggaagctt agaaaagggg aaagtaggtg ggggttggcc tccttagaga | 3120 |
| aaagttctag agtatttgaa tcttaagatc gtgctagatg tgagatgaat gggcctctca | 3180 |
| gataaataga aaaataagca agactgtgga atacattgta caaatacgga aactaatggg | 3240 |
| tatcctagaa tgaaagggaa ctgcatatgg ccaagaaaag gataagaagt aaactgagtt | 3300 |
| agattatagg gggtcagtgt tttgttcaga aactaatact cctcggaaaa ttccagtttc | 3360 |
| ttcaagtatg aagttgctgt caggaattgt gttgacttga gccaagagtc tgtgctactc | 3420 |
| atgagcatgc ccctttataa gtgacgtccc tctgttttat tagcctcctt gcttctgtat | 3480 |
| gtttcagatg tgacttccta acc | 3503 |

### BRIEF DESCRIPTION OF THE FIGURES

FIG. 1. Equiluminant surround input improves contrast encoding in human subjects.

a) Schematic of the experimental procedure. A 2-alternative forced-choice task was used to measure contrast sensitivity thresholds. Subjects were asked to fixate a spot placed in the center of the CRT monitor. Stimuli were either a temporally or spatially modulated circular stimulus presented on either side of the fixation spot. Subjects were asked to indicate on which side the stimulus was presented. Their ability to detect the stimulus was determined by their forced-choice response. Unless otherwise noted, stimuli were 1° in diameter at 6° eccentricity with a 4 Hz sinusoidal temporal modulation.

b) Temporal contrast sensitivity in normal vision subjects (n=8) was reduced by a low luminance (0.15 cd/m²) vs luminance matched (27.7 cd/m²) surround. The mean luminance of the target stimulus was not varied across the two conditions.

c) Acuity at a fixed contrast (40%) is improved by an equiluminant surround in normal vision subjects. At the eccentricity tested (5°), the surround almost doubled the acuity threshold, equivalent to a 0.52-to-1.03 shift on the logMAR scale (n=6). Error bars denote standard deviation.

d) Expanding a black annulus around the 1° stimulus lead to a cumulative degradation of contrast encoding (n=9). This effect of the surround decays in proportion to the distance from the center.

e) Adding an equiluminant annulus around a stimulus placed on a low luminance background improved contrast sensitivity. Using a small, 0.3° diameter stimulus and a range of annuli widths revealed that contrast sensitivity only improved beyond a minimal annulus width (indicated by arrows). For stimuli placed closer to the fovea, this function shifted upward and leftward (0.3° eccentricity and 2° eccentricity), consistent with smaller receptive field size (n=6).

f) Schematic of retinal circuits. Horizontal cell (HC) mediated feedback circuits are labelled HC. S- M- and L-cone photoreceptors and rod photoreceptors are shown. CBC: cone bipolar cell, RBC: rod bipolar cell, All: All amacrine cell, GC: ganglion cell.

g) Schematic showing the contributing photoreceptor types in various genetic conditions. Subjects with different genetic conditions were tested to identify the cell types contributing to the surround effect. The conditions tested included: normal vision subjects (all photoreceptors functioning), achromatopsia (rod-only vision), blue cone monochromatism (BCM, rod and S-cone vision), congenital stationary night blindness (CSNB, cone-only vision), Bornholm eye disease (BED, lack of one cone opsin; M-opsin for two of three patients tested, L-opsin for one patient). In patients with BED, silent substitution allowed isolation of each photoreceptor type. See Example 1 for more details.

h) Unlike for normal vision subjects (n=8, top trace), an equiluminant surround provided only a marginal improvement over a mismatched surround in patients with achromatopsia (n=5, first, second, third, fourth and sixth traces from bottom in low luminance surround) and blue cone monochromatism (n=3, second, third and fifth traces from top in low luminance surround), indicating an essential role for cone photoreceptors in mediating the effect.

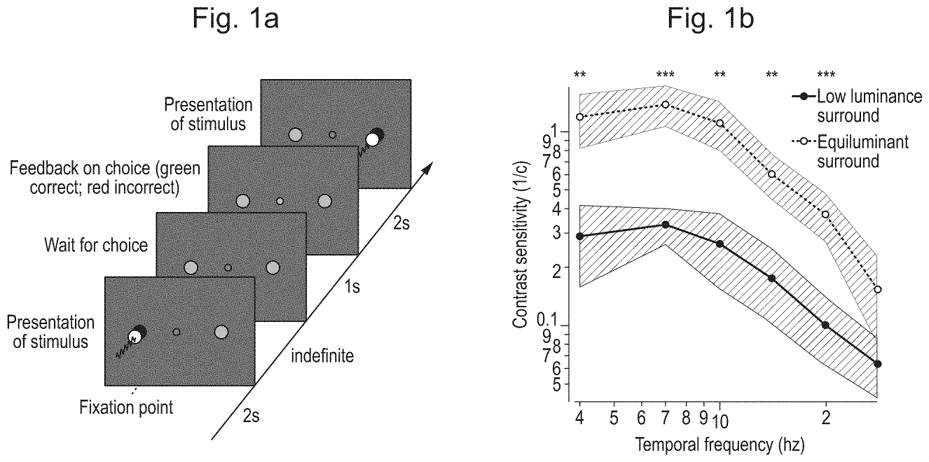

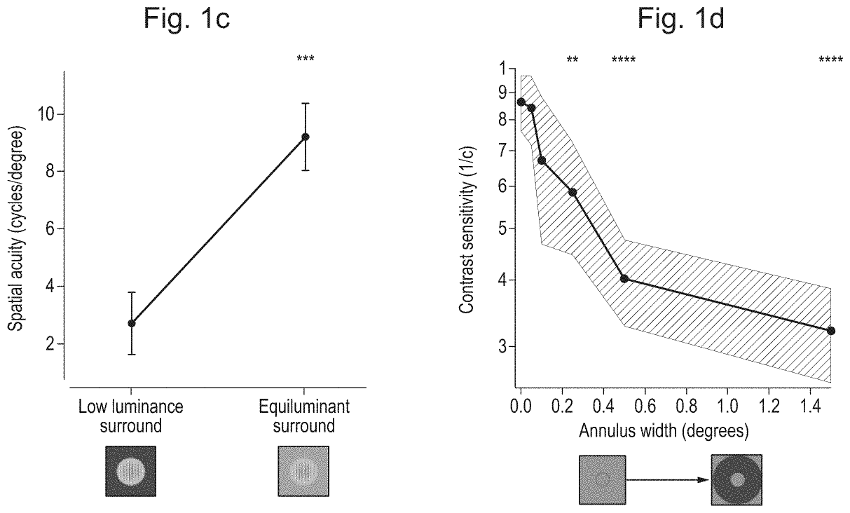

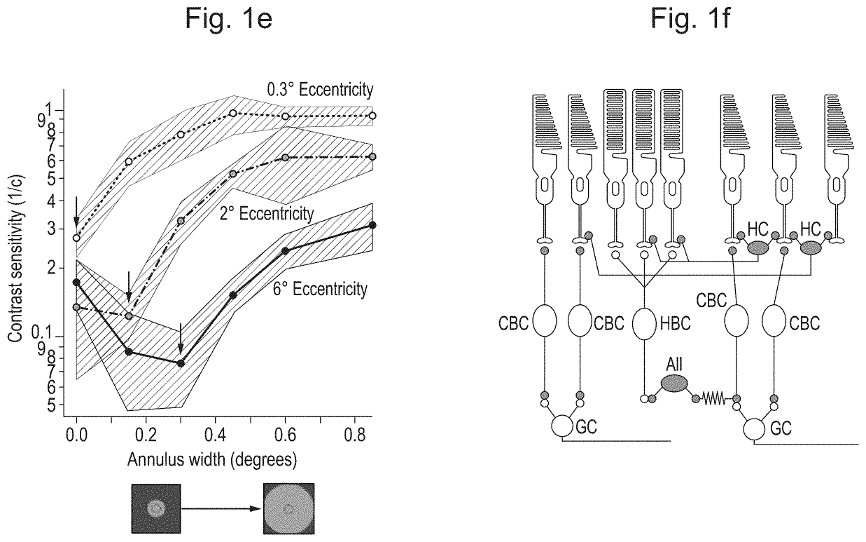

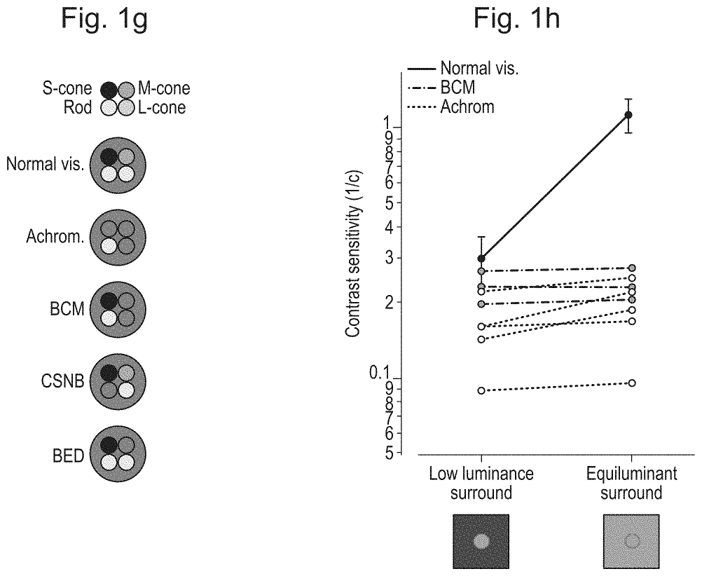

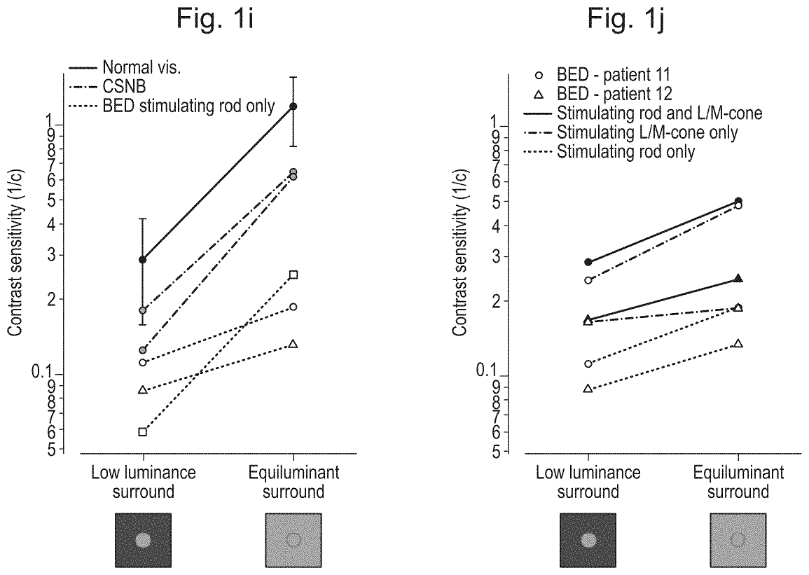

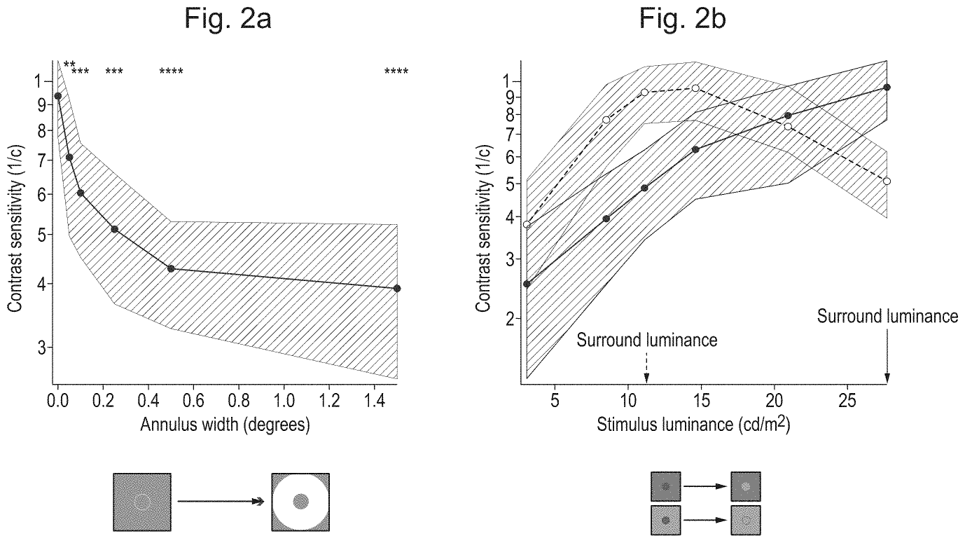

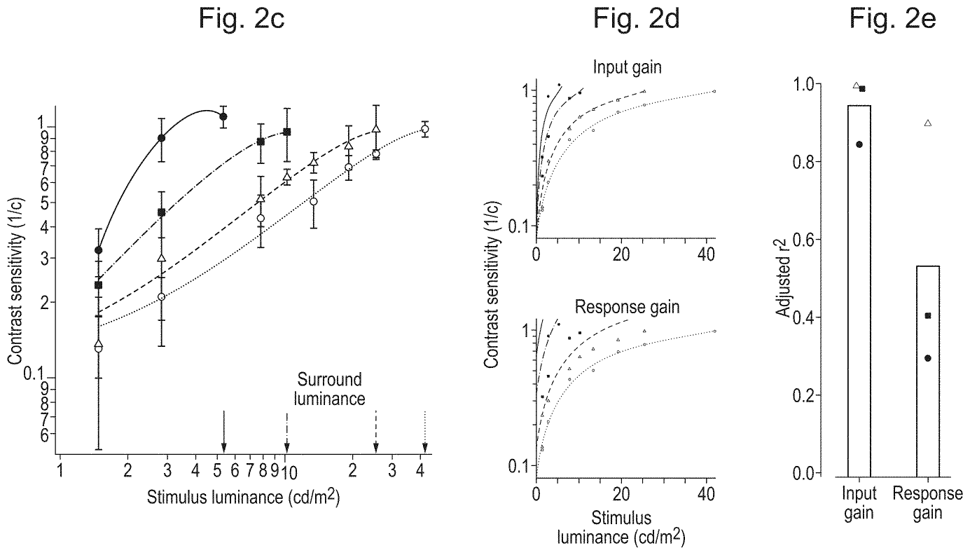

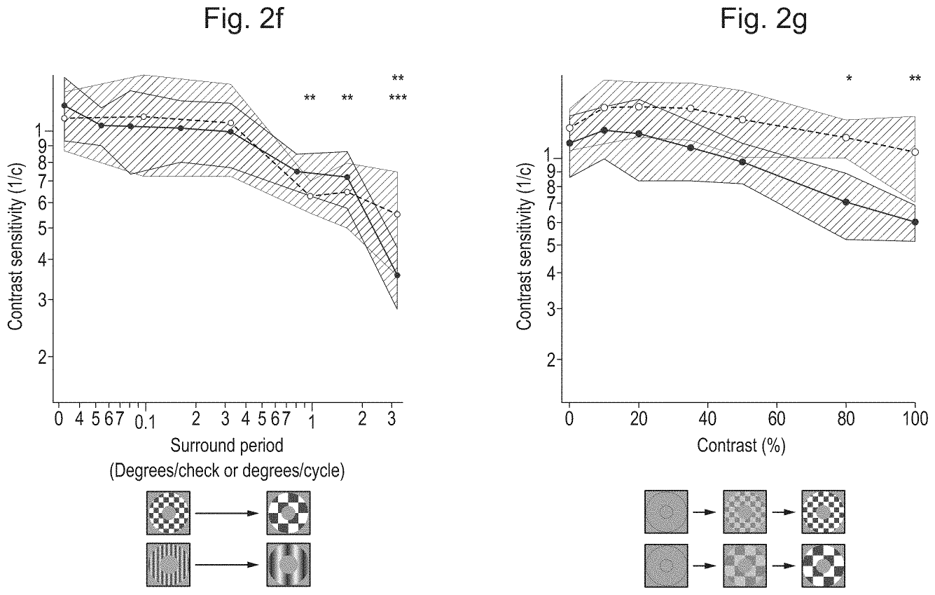

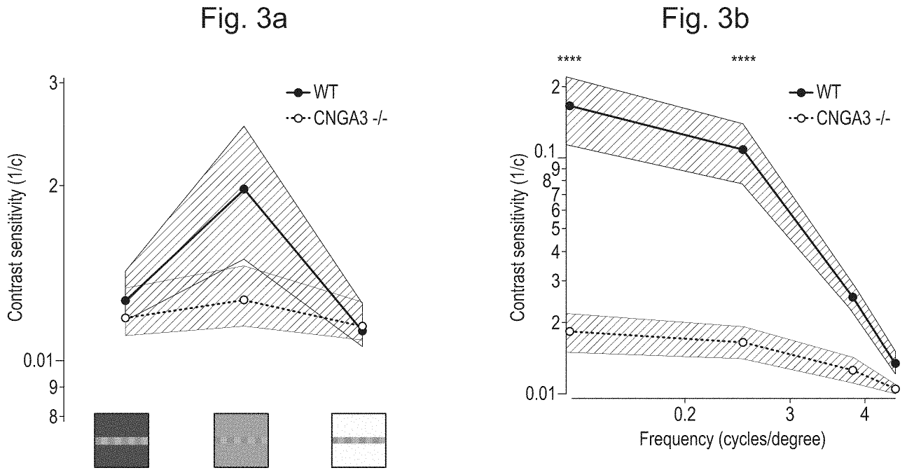

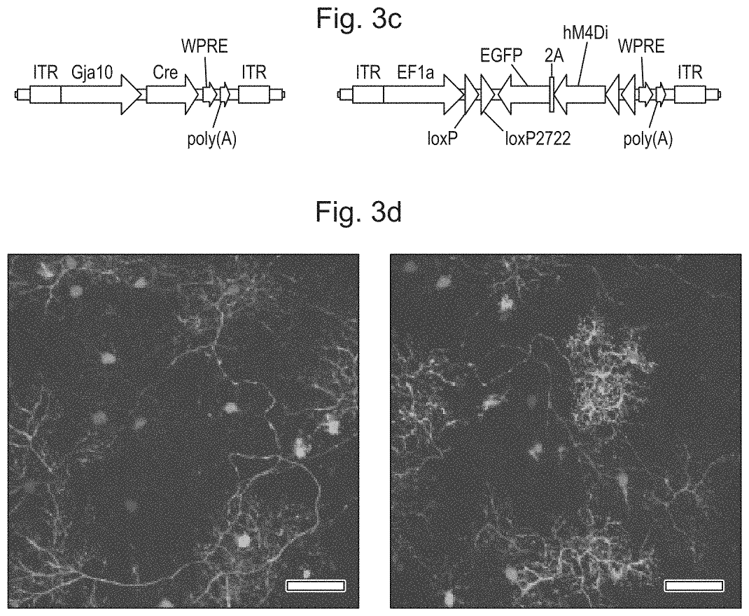

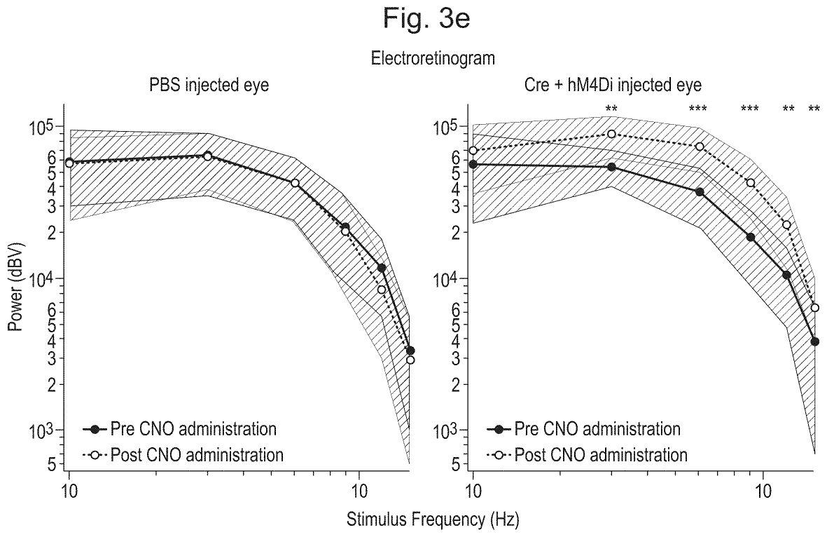

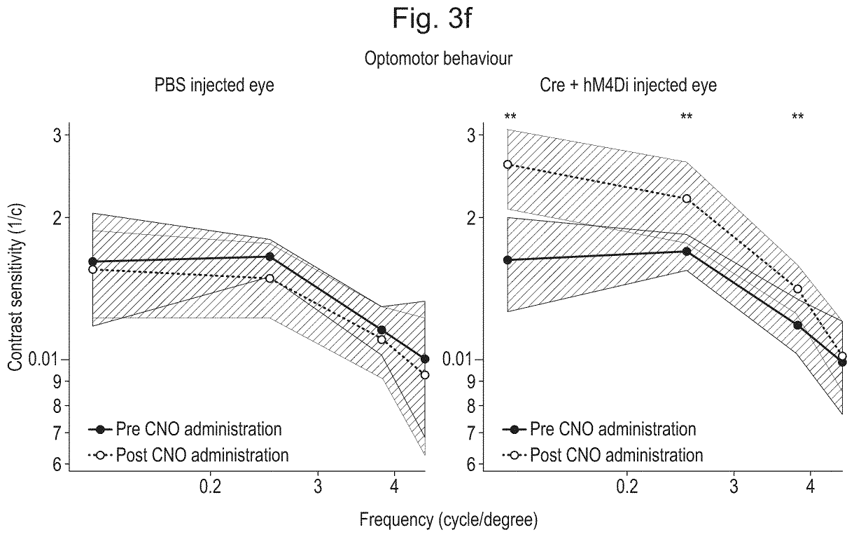

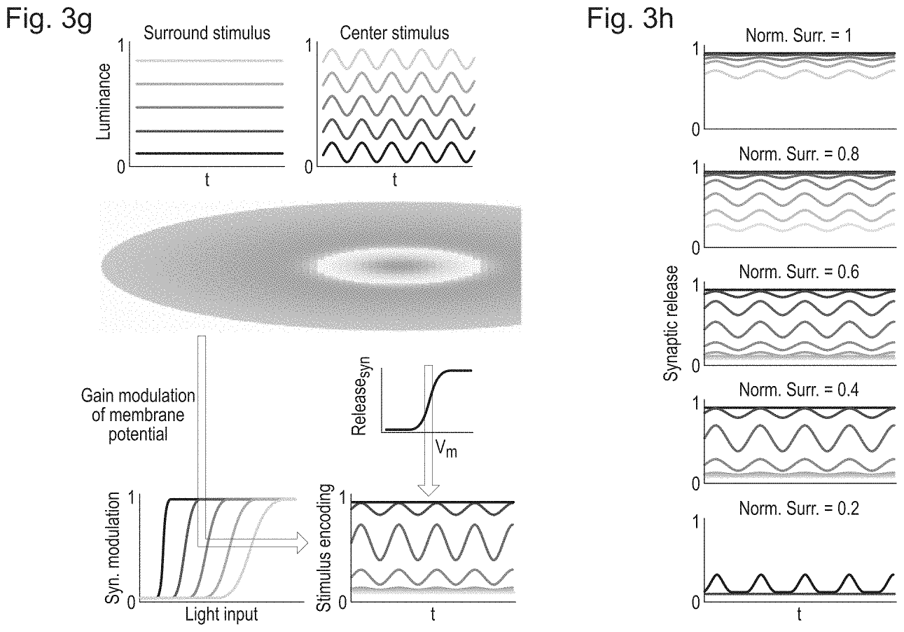

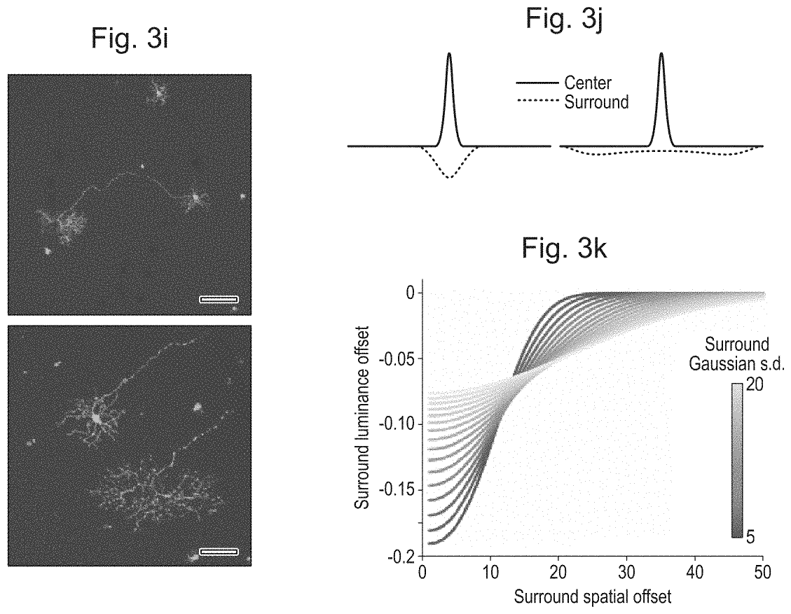

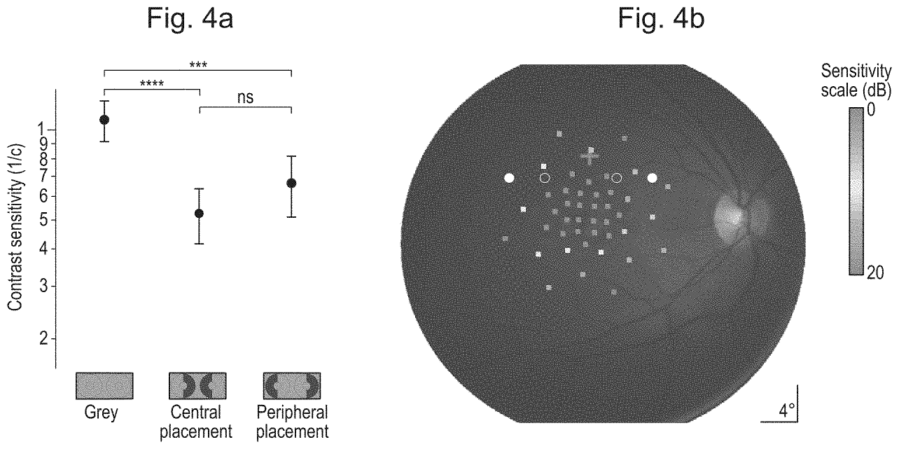

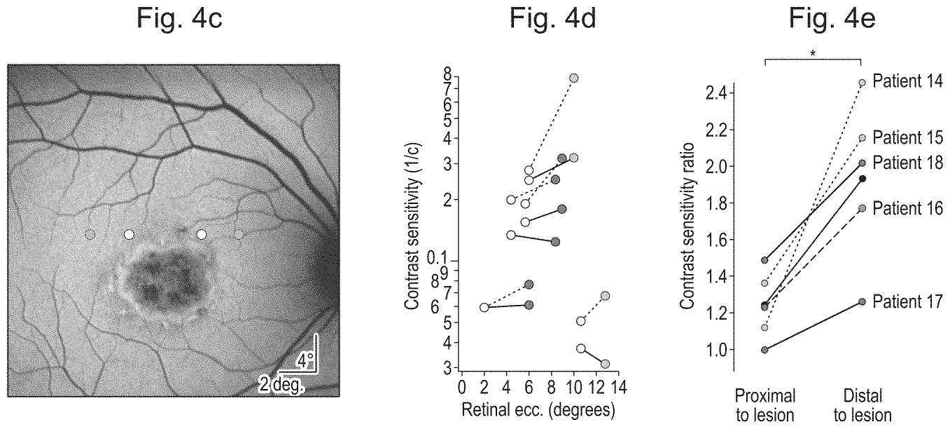

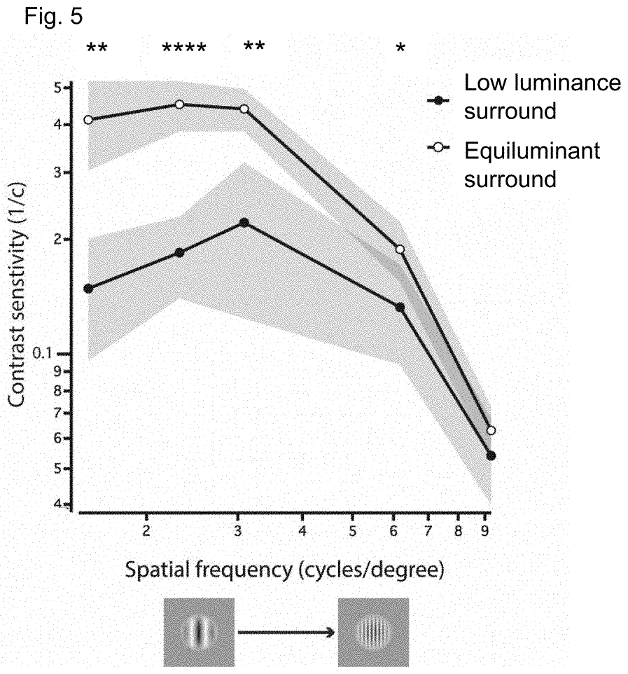

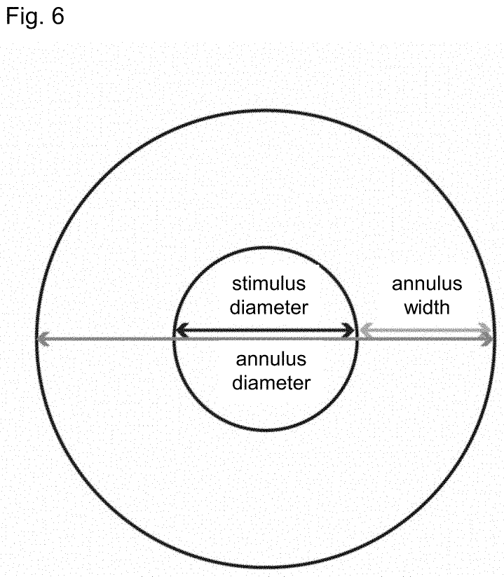

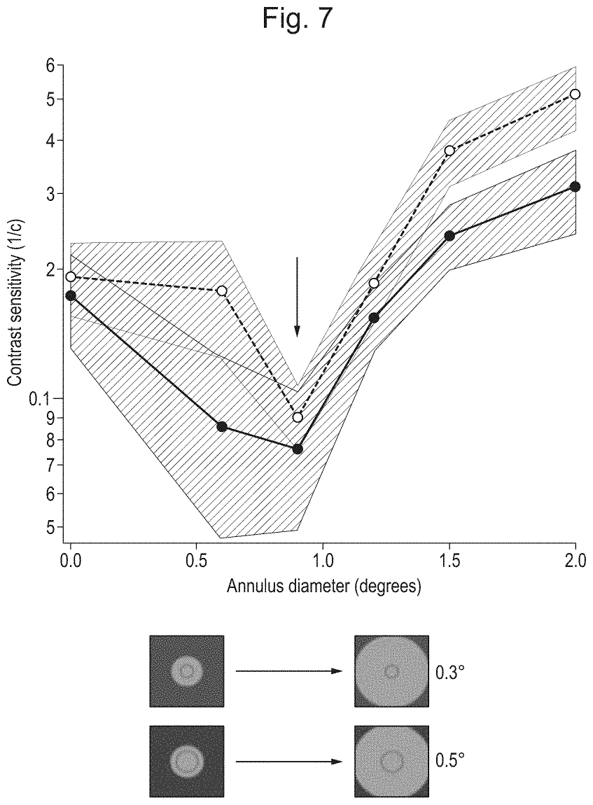

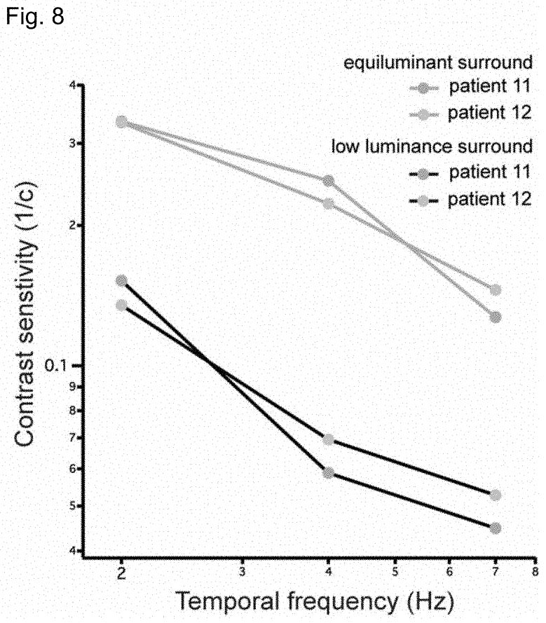

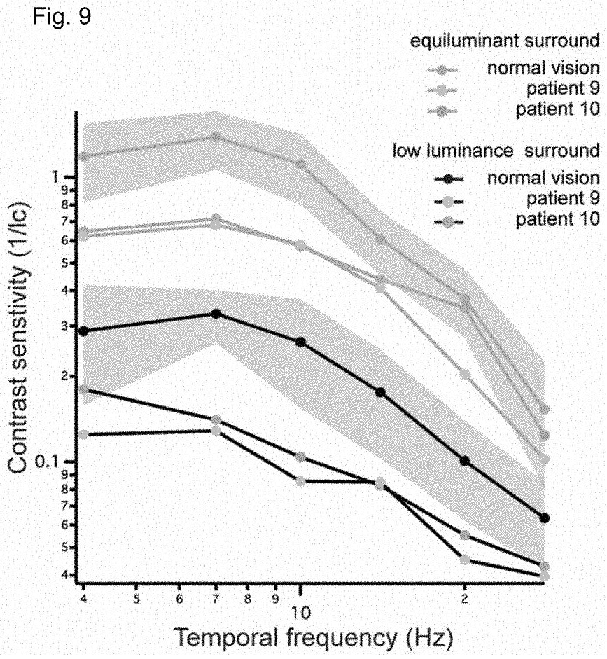

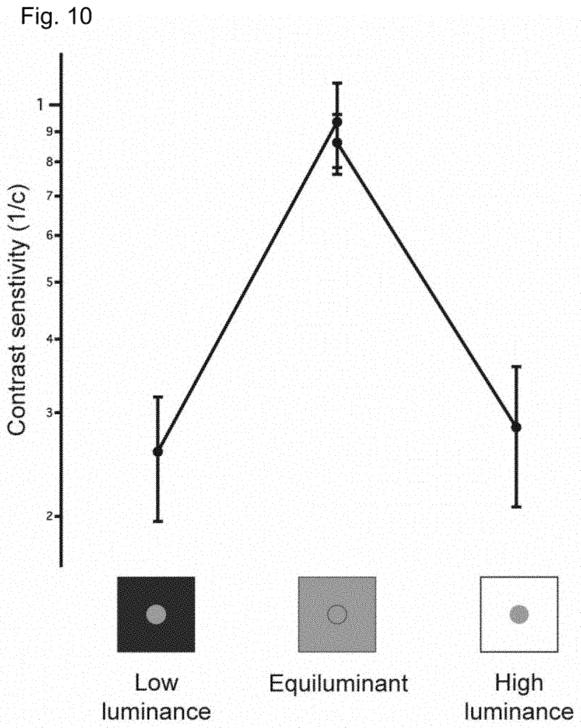

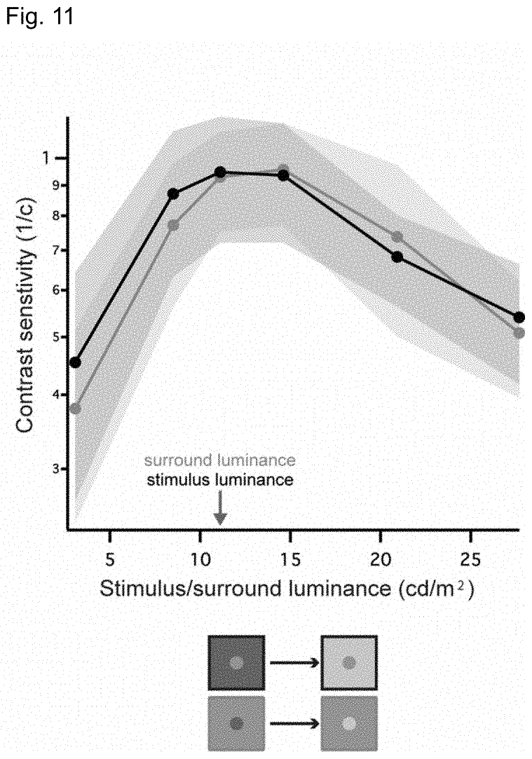

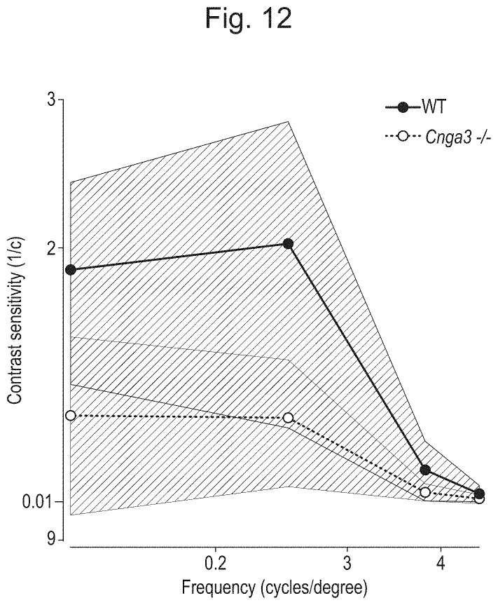

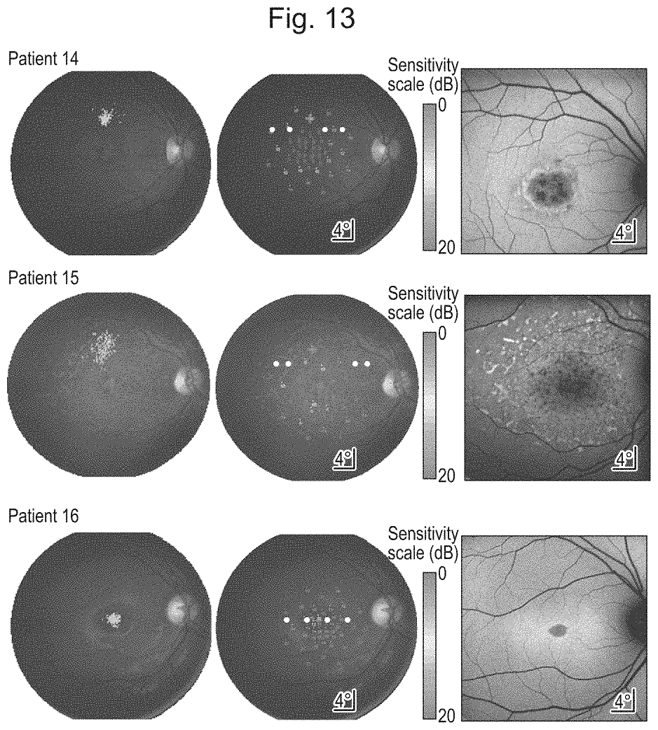

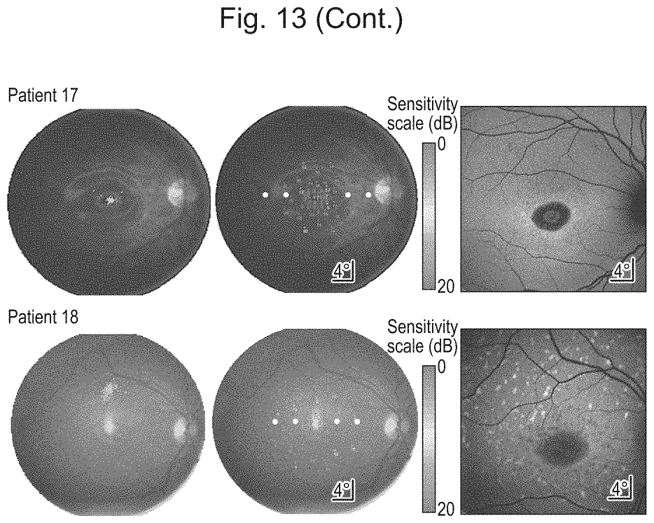

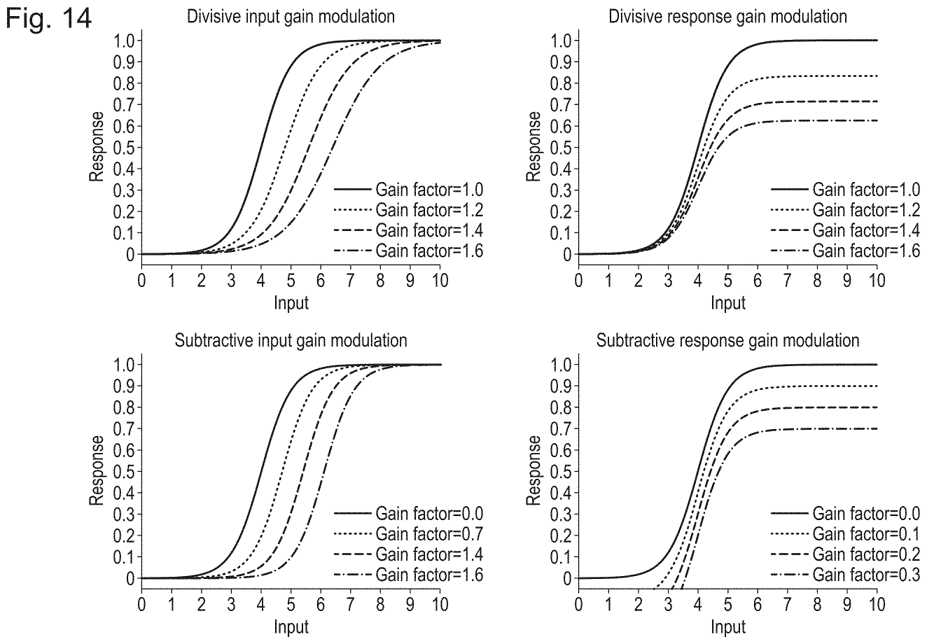

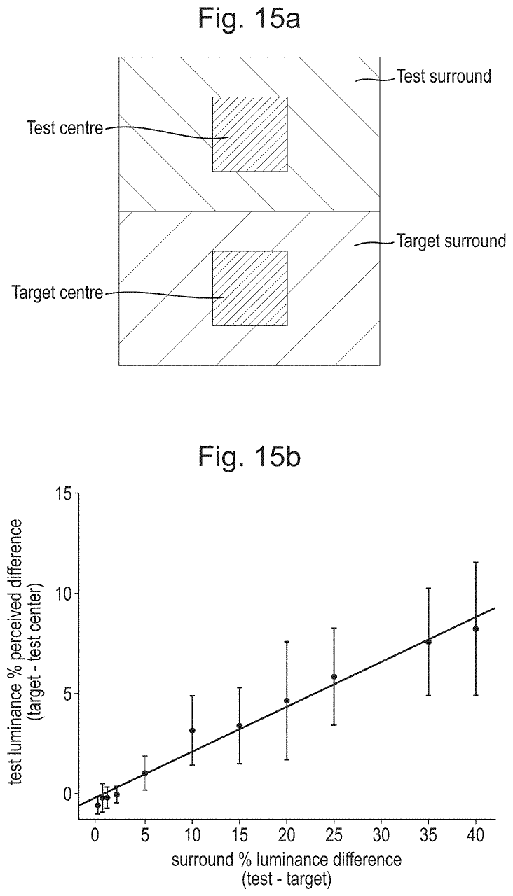

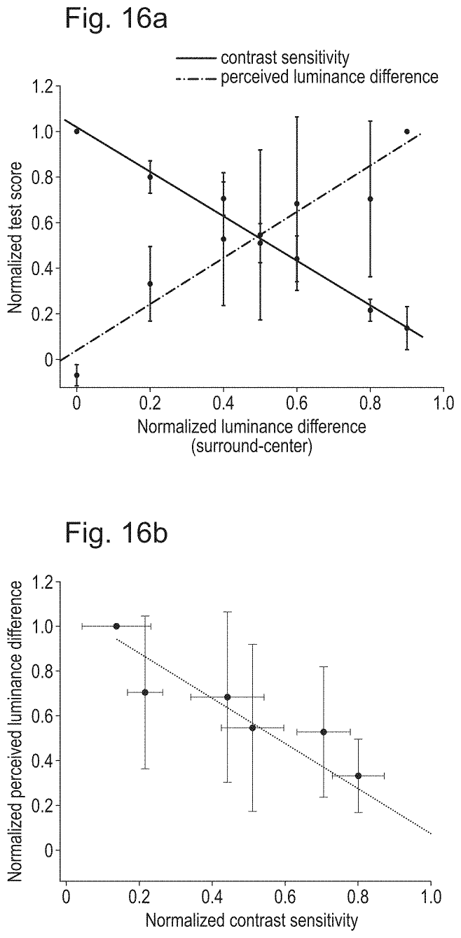

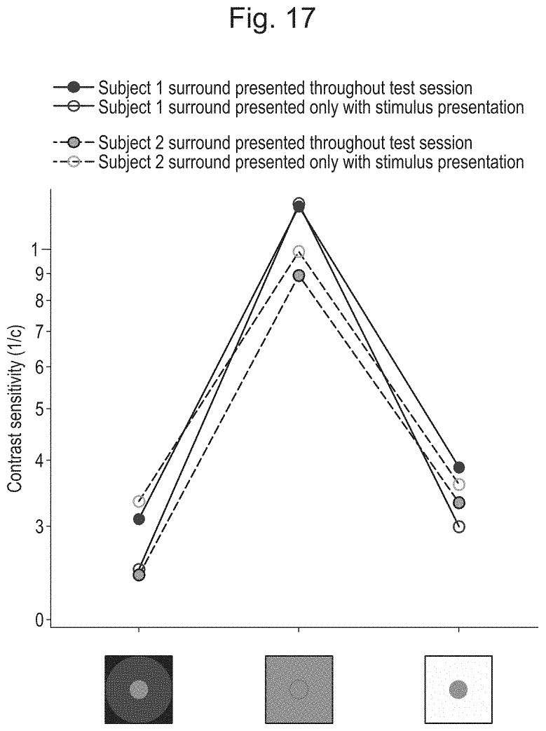

i) The surround similarly affects rod, cone and rod-cone mediated vision. Selective stimulation of rod photoreceptors in Bornholm eye disease patients (first, second and third traces from bottom in low luminance surround, n=3) showed the effect of surround luminance on rod photoreceptors is positive rather than suppressive. A similar effect was observed for cone-only mediated vision (CSNB patients, n=2, fourth and fifth traces from bottom in low luminance surround). Normal vision subjects with stimulation of both rod photoreceptors and cone photoreceptors are shown in the top trace for comparison (n=8, data from FIG. 1b).

j) An equiluminant surround improved vision in two Bornholm eye disease patients (data from each distinguished by triangular and circular markers) for rod only (first and second traces from bottom in low luminance surround), cone only (third and fifth traces from bottom in low luminance surround) and rod-cone mediated vision (first and third traces from top in low luminance surround). Co-stimulation of both rod photoreceptors and cone photoreceptors did not lead to interference but to the highest contrast sensitivity. All contrast sensitivity values are calculated as 1/contrast threshold (1/c). Values shown are mean averages and shaded regions denote standard deviations, p-values were calculated using a one-way ANOVA, with Tukey's post hoc multiple comparison test (b); a paired t-test (c) or a one-way ANOVA, with Dunnett's post hoc multiple comparison test (d, h, i, j). ****p<0.0001, ***p<0.001, **p<0.01, *p<0.05.

FIG. 2. Surround mean luminance controls gain of contrast encoding.

a) Surrounding the stimulus area with a white, high luminance (64 cd/m²) annulus reduced contrast sensitivity similarly to a low luminance annulus. The effect was also cumulative and a thin edge (0.05° annulus width) was not sufficient to reproduce the entire effect (n=9).

b) Temporal contrast sensitivity threshold was improved by increasing the mean luminance of the stimulus area until it matched the luminance of the surround (lower trace at y-intercept). Shifting the surround luminance to a lower value (arrow at position 11 on x-axis) also shifted the peak contrast sensitivity (curved, upper trace at y-intercept). The most sensitive encoding occurred when the stimulus and the surround were equiluminant, and any difference in luminance between the stimulus and the surround (either higher or lower) reduced the sensitivity of contrast encoding (n=10).

c) The effect of several surround luminance values (shaded arrows) were compared for a range of mean stimulus luminance values. Contrast encoding functions (corresponding shades) were shifted rightwards with increasing surround luminance, but maximum contrast sensitivity remained comparable (n=4).

d) Using the polynomial fit from one set of data (collected with the surround set to the highest luminance) computations were used to predict fits for the remaining three sets of data. An input gain computation captured the shift between luminance conditions better (top graph) than a response gain computation (bottom graph).

e) Comparison of r² values for response and input gain computed fits (r²=0.94 and r²=0.53, respectively).

f) An equiluminant annulus with high intrinsic contrast showed a suppressive effect, in a manner dependent on the spatial frequency of the pattern within the annulus (n=8 for checker—lower starting trace at 0 degrees/cycle; n=6 for sine—upper starting trace at 0 degrees/cycle). Low period, high-frequency patterns were not different from grey in the way they affected encoding, while lower frequencies caused a reduction of contrast sensitivity.

g) Consistent with this, only an annulus with a low spatial frequency pattern (0.97°/check) and a high intrinsic contrast affected encoding (black trace, n=8; starred values) while an annulus containing a higher frequency pattern (0.32°/check) did not (top trace, n=8). Stars denote significance compared to 0% contrast surround. All contrast sensitivity values are calculated as 1/contrast threshold (1/c). Values shown are mean averages and shaded regions denote standard deviations, p-values were calculated using a one-way ANOVA, with Dunnett's post-hoc multiple comparison test. ****p<0.0001, ***p<0.001, **p<0.01, *p<0.05.

FIG. 3. Improved vision following restoration of lateral input to rod photoreceptors.

a) Surround luminance affects the encoding of contrast in mice. Optomotor behaviour was driven by a drifting sinusoidal pattern of 1° height placed in the center of the computer screens. During stimulus presentation, the rest of the screen was either set at low-luminance (left-hand box), equiluminant (middle box) or high-luminance (right-hand box). During inter-trial intervals, the screens were set at a middle box level. In the equiluminant (middle box) condition, wild-type mice (top trace, n=9) showed increased contrast sensitivity compared to the low- and high-luminance conditions, whereas Cnga3^{-/-} mice (bottom trace, n=9) did not. These results are analogous to those in normal vision human subjects and patients with achromatopsia (see FIG. 2a).

b) Contrast sensitivity was measured with full-screen grating stimuli for a range of spatial frequencies. Contrast sensitivity was similarly reduced in Cnga3^{-/-} mice (n=9) compared with wild-type mice (n=5) at all values tested.

c) Schematic of AAV constructs used to drive expression of hM4Di in horizontal cells. One AAV vector contained a Gja10 promoter driving Cre while the other AAV vector contained a flexed hM4Di and GFP cassette.

d) Example images of floxed transgene expression in horizontal cells in the mouse retina (whole-mount, scale bar=50 μm).

e) Chemogenetic activation of horizontal cells improves outer retina function. Fourier analysis (see Example 1) of electroretinogram responses for stimuli of different temporal frequencies at 0.1 cd/m². Tests were repeated before and after intraperitoneal injection of the hM4Di activator CNO. Data for sham-PBS and hM4Di injected eyes are shown in the left and right panels respectively. No significant difference was observed in the PBS injected eyes (left panel, n=8), while a significant improvement in frequency modulation was observed in hM4Di-injected eyes (right panel, top trace, n=8).

f) Optomotor behavior was used to measure contrast sensitivity in eyes following the injection of hM4Di or PBS solution. Tests were repeated before and after intraperitoneal injection of the hM4Di activator CNO. No significant difference was observed in PBS injected eyes (left panel, n=9), while a significant improvement in contrast sensitivity was observed in hM4Di-injected eyes (right panel, n=9).

g) A simple graded release version of an “integrate and fire” model captures the behaviour of the effect reported here. A center-surround stimulus with a temporally modulated center (top right graph, oscillating traces) and an unmodulated surround (top left graph, horizontal traces) were presented to the model neuron. A difference of Gaussian's receptive field model was used to simulate spatial sampling of the stimulus. The divisive surround component of the receptive field scaled the encoding function along the input axis (bottom left graph, S-curve traces).

h) As a result, maximum modulation is proportional to surround luminance and different stimuli are best encoded for a given surround luminance (one condition also shown in bottom right panel g).

i) sparse labeling of horizontal cells with an AAV2/2 vector demonstrates lateral displacement of dendrites and axon terminal (scale bars: 50 μm top panel, 25 μm, bottom panel).

j) Left panel: schematic of a classic ‘difference of Gaussians’ model for center-surround organization with aligned peaks. Right panel: alternative model including a shift between the peaks of the center and surround distributions,

k) In a classic model of center-surround receptive fields with overlapping Gaussian distributions, the center largely determines its own gain. Introducing a spatial offset, mirroring the anatomy, allows the surround to set a gain that more accurately represents the context. Gaussian distributions with different standard deviations were tested (identified by different saturation of shading). For all conditions, a shift between center and surround improved context representation. All contrast sensitivity values are calculated as 1/contrast threshold (1/c). Values shown are mean averages and shaded regions denote standard deviations, p-values were calculated using a one-way ANOVA, with Tukey's post-hoc multiple comparisons test (a, b) or with a two-way ANOVA, with Sidak's post-hoc multiple comparisons test (e, f). ****p<0.0001 ***p<0.001, **p<0.01, *p<0.05.

FIG. 4. Impaired lateral input affects the remaining vision in macular degeneration.

a) A unilateral virtual ‘scotoma’ affects contrast encoding in normal vision subjects. A half annulus with a 4° width placed either more central or more peripheral to the stimulus significantly worsened contrast sensitivity thresholds (n=6, error bars denote standard deviation).

b) Microperimetry allowed functional mapping of the degenerating area in patients with Stargardt disease. Microperimetry scores are shown here superimposed on a fundus reflectance image. Color indicates the sensitivity scores in decibels, dB, for each location. The cross indicates the average fixation location. This measurement guided the placement of stimuli to allow comparison of contrast sensitivity at a location proximal to the lesion with a location more distal to the lesion (indicated on the image as open and filled white circles, respectively). Placement of the stimuli was different for each patient tested.

c) Short Wavelength (486 nm) Fundus Autofluorescence imaging of the retina showing the appearance and extent of the macular lesion for the same patient shown in panel (b). Open and filled circles indicate the proximal and distal locations tested. Corresponding patient data is visible as the top two traces in panel d and is marked as patient 14 in panel e.

d) Data for five patients with Stargardt disease. Open circles indicate contrast thresholds measured at locations proximal to the lesion; closed circles indicate contrast thresholds measured at locations distal to the lesion. Black lines join the measurements made with a low luminance surrounds; grey lines join measurements made with an equiluminant surround.

e) Data from d expressed as a ratio of contrast sensitivity for equiluminant over low luminance surrounds for each patient at each location. The ratio of contrast sensitivities in equiluminant vs low luminance surrounds was significantly greater for stimuli placed distally from the degenerate area, p-values were calculated using a one-way ANOVA, with Dunnett's post-hoc multiple comparisons test (a) or a paired f-test (e). ****p<0.0001 ***p<0.001, **p<0.01, *p<0.05.

FIG. 5. An equiluminant surround improves spatial contrast sensitivity functions.

Gabor patches of different spatial frequencies were presented inside a 1° stimulus area, surrounded by a 1.75° annulus width (n=9). All contrast sensitivity values are calculated as 1/contrast threshold (1/c). Values shown are mean averages and shaded regions denote standard deviations, p-values were calculated using a one-way ANOVA, with Tukey's post hoc multiple comparison test. ****p<0.0001, ***p<0.001, **p<0.01, *p<0.05.

FIG. 6. Schematic showing “stimulus diameter,” “annulus diameter” and “annulus width”.

Annulus width was defined as (annulus diameter−stimulus diameter)/2.

FIG. 7. Anatomical limit of the annulus effect is independent of the stimulus size.

Surround effect minimum diameter suggestive of a displaced hard wired origin. A small diameter stimulus (0.3° diameter, bottom trace) and a larger diameter stimulus (0.5° diameter, top trace) were used to map the geography of the annulus effect at a 6° eccentricity (n=6). The contrast sensitivity threshold was measured with equiluminant annuli of increasing width. Note that the curve obtained with the larger stimulus is shifted upwards relative to the curve obtained with the smaller stimulus. The lowest contrast sensitivity for both stimuli was seen with an annulus of the same width (indicated by an arrow). Further increasing annulus width improved contrast sensitivity for both stimuli at a similar rate. Values shown are mean averages and shaded regions denote standard deviations.

FIG. 8. Contrast sensitivity in rod-only mediated vision is affected by surround luminance

Temporal contrast sensitivity functions for two patients (top and second from bottom traces at a temporal frequency of 7 Hz and second from top and bottom traces at a temporal frequency of 7 Hz) with Bornholm Eye Disease. Silent substitution allowed selective stimulation of rod photoreceptors. For both patients, contrast sensitivity was worsened by a low luminance (bottom two traces) vs equiluminant (top two traces) surround at all tested frequencies.

FIG. 9. Contrast sensitivity in cone-only mediated vision is affected by surround luminance

Temporal contrast sensitivity functions for two patients with Congenital Stationary Night Blindness (CSNB, bottom and third from top traces and second from top and second from bottom traces). For both patients, contrast sensitivity was improved by an equiluminant (grey connecting lines) vs low luminance (black connecting lines) surround. Comparative data for normal vision subjects is shown (top trace and third from bottom trace, n=8, values shown are mean averages, standard deviation indicated by shading, data as shown in FIG. 1 b).

FIG. 10. Comparable effects of high and low luminance suggestive of adaptive mechanism

Comparison of temporal contrast sensitivity with low luminance, equiluminant and high luminance surrounds in normal vision subjects. Contrast thresholds were measured using a 1° stimulus placed at a 6° eccentricity, with a 4 Hz temporal sinusoidal modulation. The effect of a low or high luminance surround was tested on the same subjects but in separate testing sessions (n=9). The equiluminant condition was repeated for each session. Values shown are mean averages; error bars denote standard deviation.

FIG. 11. Surround luminance tunes contrast encoding properties of the stimulus

Peak contrast sensitivity determined by the difference between center and surround luminance. Top trace at y-intercept: temporal contrast sensitivity thresholds were measured with a range of surround luminance values (n=9). High sensitivity was obtained when the surround luminance was equiluminant to the mean stimulus luminance (arrow) and progressively degraded with mismatched luminance levels. Bottom trace at y-intercept: the experiment was repeated with a range of mean stimulus luminance values with the surround luminance set to a single value (arrow, data as in FIG. 2b). Values shown are mean averages and shaded regions denote standard deviations.

FIG. 12. Contrast sensitivity functions obtained with a modified version of the optomotor behavioural paradigm

Contrast sensitivity functions could be obtained for wild-type (n=4, top trace) and Cnga3^{-/-} mice (n=4, bottom trace) using a 1° height sinusoidal stimulus. As expected, contrast sensitivity values were reduced compared to those obtained with full-screen presentation of the same stimuli (FIG. 3b). Values shown are mean averages, shaded regions denote standard deviations.

FIG. 13. Microperimetry data and Fundus Autofluorescence images for individual Stargardt disease patients

Microperimetry data and Fundus Autofluorescence images for all tested Stargardt disease patients (n=5). Left column: fixation stability (scattered shading, see Example 1) superimposed on a fundus image of the eye. Fundus Autofluorescence data was obtained prior to microperimetry. Middle column: sensitivity values obtained from microperimetry superimposed on fundus photograph of the eye. Bar indicates sensitivity in decibels. The cross indicates the average fixation spot for images in the left and middle column. Superimposed white circles indicate stimulus size and locations used for psychophysical testing of contrast sensitivity proximal (central placement) and distal (peripheral placement) to the lesion. Right column: shortwave-length (486 nm) Fundus Autofluorescence images. The area of atrophy presents with a decreased signal in all patients.

FIG. 14. Mathematical models of gain and their predicted effect on input-output functions.

Schematic showing input-output functions at different gain values (indicated by corresponding shade), when the gain is implemented through a division of, or a subtraction from the input (left top and left bottom panels respectively) or by the division of or subtraction from the response (right top and right bottom panels respectively). Implementing the gain through a divisive mechanism (top panels) scales the function, whereas implementation through a subtractive mechanism shifts the function without scaling (bottom panels). When the gain factor is applied to the input the shift/scale occurs along the x-axis (left panels), and when applied to the response it occurs along the y-axis (right panels). Note that different gain factor values were applied for the different computations for illustration purposes.

FIG. 15. Measurement of altered luminance perception in the simultaneous contrast illusion

a) The magnitude of the altered luminance perception in the simultaneous contrast illusion was measured in human subjects with a template-match task. Using a cathode-ray tube monitor, subjects were presented with two sets of a uniform grey field with a smaller square placed in the center of each. The “target” set, visible in the lower half of the screen, displayed a 2% contrast difference between the center and the surround and was never altered throughout the testing session. In the upper half of the screen, the “test surround” was set to a predetermined value for each trial, ranging between 60% white (34.9 cd/m²) and 100% white (63.7 cd/m²). The subject was then asked to alter the appearance of the “test center” until it matched the appearance of the “target center”.

b) Mean average data from all test subjects (n=9) showing that increasing the luminance difference between the test and target surrounds increased the error in matching the test to target center luminance (line fit r²=0.92). Error bars denote standard deviation.

FIG. 16. Anti-correlation between contrast sensitivity and luminance perception imposed by center-surround luminance difference.

a) Effect of intrinsic contrast between center and surround on template-match and contrast sensitivity tasks. The magnitude of the error in the template-match task (line with normalized test score at position 0 on y-intercept fit, r²=0.92, data as shown in FIG. 15b) increased as contrast sensitivity (line with normalized test scope above position 1 on y-intercept fit, r²=0.98) decreased. Note that the same subjects (n=4) and the same center and surround luminance values were used for the two tasks.

b) Correlation between template-match and contrast sensitivity scores, (line fit, r²=0.88; Spearman correlation=1).

FIG. 17. Surround luminance mediates fast effects on contrast sensitivity.

Surround-mediated effects do not require prolonged adaptation to surround luminance. Comparison of temporal contrast sensitivity with low luminance, equiluminant and high luminance surrounds in two normal vision subjects (the two traces with the highest contrast sensitivity at equiluminant surrounds correspond to one subject and the two traces with the lowest contrast sensitivity at equiluminant surrounds correspond to a second subject). Contrast thresholds were measured using a 1° stimulus placed at a 6° eccentricity, with a 4 Hz temporal sinusoidal modulation. Closed circles indicate measurements made with the luminance of the background set throughout the test session. Very similar measurements were obtained (open circles) when the background luminance was set to equiluminant during the response period between stimuli presentations and only decreased or increased to coincide with stimulus onset. The similarity between the measurements obtained with these two different presentation parameters indicates that the effect of luminance in the surround is not due to slow adaptive mechanisms and that the surround provides fast feedback to the center.

### DETAILED DESCRIPTION

It is to be understood that the terminology used herein describes particular embodiments of the invention only, and is not intended to be limiting.

In addition, as used in this specification and the appended claims, the singular forms “a”, “an”, and “the” include plural references unless the stated otherwise. Thus, for example, reference to “a polynucleotide” includes “polynucleotides”, reference to “a promoter” includes “promoters”, reference to “a vector” includes two or more such vectors, reference to “a molecule” includes two or more such molecules and the like.

As described herein, the term nucleic acid molecule includes, but is not limited to a coding sequence, an expression construct, or a vector.

The terms “retinal disorders” or “retinal dystrophies” may be defined as diseases of the retina. Retinal disorders may be characterized by progressive loss of photoreceptor cells and concomitant loss of vision. Alternatively, retinal disorders may be characterized by progressive or stationary loss of photoreceptor cell function and concomitant loss of vision. Disorders or dystrophies may be inherited or acquired. In one aspect, the disorder is selected from the list comprising: achromatopsia, blue cone monochromatism (BCM), and macular degeneration (including age related macular degeneration and Stargardt disease).

The terms “patient” and “subject” may be used interchangeably. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human. The subject may be male or female. The subject is preferably identified as being at risk of, or having, one of the above-mentioned retinal disorders or dystrophies.

The terms “treat,” “treated,” “treating,” or “treatment” as used herein refer to both therapeutic treatment and prophylactic or preventative measures, wherein the object is to prevent or slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

All publications, patents and patent applications cited herein are incorporated by reference in their entirety.

Gene Product

Provided herein is a nucleic acid molecule encoding a gene product, such as a membrane protein. Any gene product that causes hyperpolarization of horizontal cells may be used in the present invention. Provided herein is a nucleic acid molecule encoding a gene product that hyperpolarizes horizontal cells. Provided herein is a nucleic acid molecule encoding a gene product, wherein expression of the gene product in horizontal cells hyperpolarizes horizontal cells. Provided herein is a nucleic acid molecule encoding a gene product, wherein expression of the gene product in horizontal cells hyperpolarizes horizontal cells in order to improve vision. Provided herein is a nucleic acid molecule encoding a gene product, wherein expression of the gene product in horizontal cells hyperpolarizes horizontal cells in order to improve vision in a subject in need thereof. Provided herein is a nucleic acid molecule encoding a gene product, wherein expression of the gene product in horizontal cells hyperpolarizes horizontal cells in order to improve mesopic and/or photopic vision in a subject in need thereof.

For example, the membrane protein may be an ion channel, an ion pump or a G-protein coupled receptor that is endogenously expressed in human horizontal cells, or a channel, pump or receptor that is endogenously expressed in other excitable cell types that produce a net outward conductance, such as tandem pore domain ‘leak” potassium channels (K₂ₚ1-18), voltage-gated potassium channels (Kᵥ1-9), Ca²⁺ activated potassium channels (BK, SK and IK channels), inward rectifying potassium channels (e.g. Kᵢᵣ1-8), chloride channels, and ion pumps. The membrane protein may also be variants of the above-mentioned proteins, that retain the function of the wildtype protein.

In one embodiment, the hyperpolarization of horizontal cells is mediated by an ion channel. Preferably, the hyperpolarization of horizontal cells is mediated by a potassium channel. In one embodiment the potassium channel may be a tandem pore domain ‘leak” potassium channel (a K₂ₚ channel), voltage-gated potassium channel (a Kᵥ channel), Ca²⁺ activated potassium channel (a BK, SK or IK channel), or inward rectifying potassium channel (a Kᵢᵣ channel). Specific examples of potassium channels from mammalian species include a tandem pore domain ‘leak” potassium channel (K₂ₚ1-18), a voltage-gated potassium channel (Kᵥ1-9), a Ca²⁺ activated potassium channel (BK, SK and IK channels), and an inward rectifying potassium channels (e.g. Kᵢᵣ1-8). In another embodiment, the membrane protein is a chloride channels or an ion pump.

The membrane protein, such as an ion channel or ion pump, may be a wildtype protein that is overexpressed in horizontal cells, for example, after delivery in a vector of the invention. The membrane protein may also be a variant of one of the above-mentioned proteins, such as a variant that retains the function of the wildtype protein.

The membrane protein, such as an ion channel or ion pump, may be constitutively active. Alternatively, the membrane proteins, such as an ion channel or ion pump, may be an optogenetic actuator. Optogenetic actuators are light-activated or light-driven proteins that are able to generate electrochemical signals. The terms “optogenetic” and “light-sensitive” can be used interchangeably in the art.

Optogenetic actuators of the invention preferably mediate hyperpolarization of horizontal cells by mediating a potassium conductance. The optogenetic actuator may comprise a light-sensitive potassium channel-based actuator that mediates hyperpolarization of horizontal cells. The optogenetic actuator may comprise a light-controlled membrane protein linked to potassium channel. The optogenetic actuator may comprise a light-sensitive potassium channel or a photo-activated molecule linked with a potassium channel. Such optogenetic actuators have been engineered and known in the art.

For example, in one embodiment, the optogenetic actuator is a light-sensitive synthetic potassium channel. In one embodiment, the optogenetic actuator is a light-sensitive synthetic potassium channel that leads to hyperpolarization of horizontal cells. Exemplary light-sensitive synthetic potassium channels useful in the present invention include BLINK1 and BLINK2.

BLINK 1 is a blue-light-induced K+ channel 1, and is described in Cosentino et al., 2015 (“Engineering of a light-gated potassium channel” Science, Vol. 348, Issue 6235, pp. 707-710). BLINK1 was engineered by fusing the plant LOV2-Jα photosensory module to the small viral potassium channel Kcv. BLINK1 exhibits biophysical features of Kcv, including potassium selectivity and high single-channel conductance but reversibly photoactivates in blue light. Opening of BLINK1 channels hyperpolarizes the cell to the potassium equilibrium potential.

BLINK2 is an optimised version of BLINK1, as disclosed in Alberio et al., 2018 (“A light-gated potassium channel for sustained neuronal inhibition”. Nat Methods 15(11):969-976). In particular, BLINK2 showed higher surface expression in neurons compared with that of BLINK1, as well as efficient inhibition of firing in three animal models: zebrafish, rat and mouse. Unique to BLINK2 is its post-illumination activity, which lasts tens of minutes. This property is advantageous for achieving long neuronal inhibition without toxic exposure to prolonged illumination, for instance, in the case of neuropathic pain or in behavioural animal experiments. Thus, in a preferred embodiment, the light-sensitive synthetic potassium channel is BLINK2, or a variant thereof.

In another embodiment, the optogenetic actuator may comprise a two-component optical silencer system. In another embodiment, the optogenetic actuator may comprise a two-component optical silencer system comprising photoactivated adenylyl cyclases (PACs) and the small cyclic nucleotide-gated potassium channel SthK (PAC-K actuators). PACs are blue-light-activated cyclases consisting of a blue light receptor using flavin (BLUF) domain coupled via a linker to the cyclase domain. They were identified in various species, providing a range of light sensitivities and enzyme kinetics. Suitable PACs include those described in Bernal Sierra et al., 2018 (“Potassium channel-based optogenetic silencing”. Nat Commun 9, 4611), including NgPAC1, bPAC, TpPAC, OaPAC and lc-PAC. Each of the aforementioned PACs may be used in combination with SthK in the present invention to provide a light activated increase in potassium conductance. In particular, any of SthK-NgPAC1, SthK-bPAC, SthK-TpPAC, SthK-OaPAC and SthK-lc-PAC may be used as an actuator in the present invention.

Thus, provided herein is an expression construct comprising a promoter operably linked to nucleic acid molecule encoding an optogenetic actuator comprising a potassium channel. In a preferred embodiment, provided herein is an expression construct comprising a promoter operably linked to nucleic acid molecule encoding an optogenetic actuator comprising a light-sensitive potassium channel. In a preferred embodiment, provided herein is an expression construct comprising a promoter operably linked to nucleic acid molecule encoding an optogenetic actuator comprising a photo-activated molecule linked with a potassium channel.

In a preferred embodiment, the optogenetic actuator is selected from BLINK2, BLINK1, or a photoactivated adenylyl cyclases (PACs) linked to a small cyclic nucleotide-gated potassium channel, such as NgPAC1, bPAC, TpPAC, OaPAC, lc-PAC.

Also provided is an expression construct comprising a promoter operably linked to nucleic acid molecule encoding a variant of one of the aforementioned optogenetic actuator, wherein the variant optogenetic actuator functions to mediate potassium conductance.

Expression of said optogenetic actuator in horizontal cells mimics normal, cone originating light input to horizontal cells. Alternatively, the membrane protein, such as an ion channel or ion pump, may be a chemogenetic actuator, meaning that they are engineered receptors that are activated or inhibited by synthetic ligands to alter cellular signal transduction. Expression of said chemogenetic actuator in horizontal cells and administration (systemic or local) of said synthetic ligand causes hyperpolarization of the horizontal cells.

When the gene product is an optogenetic actuator, expression of said gene products mimics the input from cone cells. Thus expression of a light-sensitive membrane protein reproduces a natural mechanism, wherein horizontal cells hyperpolarise in response to light and dynamically retune the postsynaptic photoreceptors to the increased light level. When the gene product is a chemogenetic actuator or a constitutively active protein, activation create a localized area where the range of optimal light sensitivity is statically higher, thereby improving mesopic and photopic vision.

Also provided herein is an expression construct comprising a promoter operably linked to nucleic acid molecule encoding a chemogenetic actuator comprising a potassium channel.

The gene product may be a receptor that has been linked to an ion channel or ion pump, wherein activation or inactivation by endogenous or exogenous molecules leads to hyperpolarization of horizontal cells. For example, the gene product may be a chemosensitive receptor linked to a potassium channel. Suitable receptors include G-protein coupled receptors (GPCRs), such as neuronal GPCRs. GPCRs engineered to only respond to a specific biologically inert chemical (a designer drug), are known in the art. Exemplary modified GPCRs are known as Designer Receptors Exclusively Activated by Designer Drugs (DREADDs). According to one aspect, DREADD-free neurons (for example, horizontal cells) are unresponsive to the designer drug while cells expressing the DREADD respond robustly to low concentrations of the designer drug. Therefore, it is envisaged that horizontal cells may be modified to express an engineered GPCR, such as a DREADD.

Suitable designer receptors include hM3Dq (a modified form of the human M3 muscarinic (hM3) receptor which can be activated by the inert clozapine metabolite clozapine-N-oxide (CNO)), hM4Di (a modified form of the human M4 muscarinic (hM4) receptor which can be activated by the inert clozapine metabolite clozapine-N-oxide (CNO)) or KORD (a modified form of the human kappa opioid (KOR) receptor which can be activated by the inert compound salvinorin B (SALB)). The co-expression of two engineered GPCRs within the horizontal cell population may be used to control neuronal activity bi-directionally.

In one aspect, the nucleic acid molecule comprises opsin genes or variants thereof encoding light-sensitive proteins that are naturally found in microorganisms such as archaea and bacteria. Examples of suitable opsins include bacteriorhodopsins (proton pumps), halorhodopsins (ion pumps), channelrhodopsins (ion channels), and variants thereof. Also disclosed are Leptosphaeria maculans fungal opsins (“Mac”). The opsin protein may be an inhibitory opsin protein. The inhibitory opsin protein may be selected from the group consisting of: NpHR eNpHR 1.0, eNpHR 2, eNpHR 3.0, SwiChR, SwiChR 2.0, SwiChR 3.0, Arch, ArchT, Arch 3.0, ArchT 3.0, iChR, iC++, ChloC, Slow ChloC, iC1C2, iC1C2 2.0, and iC1C2 3.0.

Expression of the gene product of the invention in a horizontal cell enables said cell to be artificially hyperpolarised and/or increases the outward conductance of cell. Hyperpolarization of horizontal cells adjusts the operating range of the rod and/or cone photoreceptor synapses that they contact. Therefore, the net result of hyperpolarizing horizontal cells is that rod photoreceptors better signal under brighter illumination (i.e., in the mesopic or photopic range). Expression of the disclosed gene product in a horizontal cell may thus improve visual acuity, improve contrast sensitivity, reduce aversion to physiological light levels, and lower flicker fusion thresholds, as measured using standard means. Preferably the artificial hyperpolarization and/or increase in outward conductance in the horizontal cell is mediated by a gene product which comprises a potassium channel.

Expression Constructs

A nucleic acid molecule of the invention may be provided in the form of an expression construct. An expression construct includes a control sequence(s) operably linked to the nucleic acid molecule as described above. Thus, the expression construct allows expression of the gene product of the invention in vivo. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. These expression constructs, in turn, are typically provided within vectors (e.g., plasmids or recombinant viral vectors). Such an expression cassette may be administered directly to a host subject.

The expression constructs or vectors of the invention improve mesopic or photopic vision. “Improving” generally means improving one or more of: poor visual acuity, poor contrast sensitivity, aversion to physiological light levels, lower flicker fusion thresholds, or any other phenotype associated with loss of cone function in achromatopsia, blue cone monochromatism (BCM), macular degeneration (including age related macular degeneration and Stargardt disease) or cone-rod dystrophies.

The properties of the expression cassette or vector of the invention can also be tested using techniques based on those described in the Examples. In particular, an expression cassette of the invention can be assembled into a vector of the invention and delivered to the retina of a Cnga3^{-/-} test animal, such as a mouse, and the effects observed and compared to a control. Preferably, the control will be the other eye of the same animal which is either untreated or treated with a control vector such as one containing a reporter gene as opposed to a sequence of the invention. Contrast sensitivity may be determined using an optomotor test, as described in Examples 1 and 5. Electroretinogram (ERG) is a diagnostic test that measures the electrical activity generated by neural and non-neuronal cells in the retina in response to a light stimulus, and may thus be used to measure the function of the retina. In human subjects, psychophysical tests including those disclosed in the Examples that are designed to measure contrast sensitivity and visual acuity, and subjective measures including flicker fusion paradigms. “photophobia” or “light aversion” may also be used.

Further routine clinical measures of contrast sensitivity and acuity include Snellen charts and Pelli-Robson charts.

Expression of the expression construct of the invention in a horizontal cell enables said cell to be artificially hyperpolarised and/or increases the outward conductance of cell. Hyperpolarization of horizontal cells adjusts the operating range of the rod and/or cone photoreceptor synapses that they contact. Therefore, the expression constructs of the invention may increase the range of optimal light sensitivity in rod photoreceptors when delivered to horizontal cells, thereby improving mesopic and photopic vision.

Promoter

Disclosed herein is an expression construct comprising a promoter operably linked to a nucleic acid molecule as described above. The promoter may be constitutive or conditionally active.

The promoter will preferably be a horizontal cell-preferred or horizontal cell-specific promoter or variant thereof. Horizontal cell-preferred expression can be defined as expression that is present in horizontal cells to a greater extent than in other cell types, such as photoreceptor cells. Horizontal cell-specific expression may be defined as expression that is only present in horizontal cells, and not in other cell types. Horizontal cell-specific expression may be defined as expression that is more than about 10 times greater, 20 times greater, 50 times greater or 100 or more times greater in horizontal cell than in other cell types, especially photoreceptor cells. Expression in horizontal cells and other cell types can be measured by any suitable standard technique known to the person skilled in the art. For example, RNA expression levels can be measured by quantitative real-time PCR. Protein expression can be measured by western blotting or immunohistochemistry.

Genes that are specifically expressed in horizontal cells, and thus have promoters that allow horizontal cell-specific expression include the gap junction alpha-10 protein (Gja10).

In one aspect the promoter comprises a promoter sequence from Gja10, or fragments of the promoter sequence thereof or variants of the promoter sequence thereof that retain the function of the Gja10 promoter.

In one aspect the promoter comprises a sequence of contiguous nucleotides from the human Gja10 promoter, or variants thereof that retain the function of the human Gja10 promoter. In one aspect, the promoter comprises a contiguous sequence of at least 400 nt, 450 nt, 500 nt, 600 nt, 700 nt, 800 nt, 900 nt, 1.0 kb, 1.5 kb, 2.0 kb, 2.5 kb, 3.0 kb or 3.5 kb of the sequence of the human Gja10 promoter, which retains the function of the Gja10 promoter. In one aspect, the promoter comprises a contiguous sequence of no more than 400 nt, 450 nt, 450 nt, 500 nt, 600 nt, 700 nt, 800 nt, 900 nt, 1.0 kb, 1.5 kb, 2.0 kb, 2.5 kb, 3.0 kb or 3.5 kb of the sequence of the human Gja10 promoter, which retains the function of the Gja10 promoter. In one aspect the promoter comprises a sequence of contiguous nucleotides from the human Gja10 promoter, or variants thereof with at least 60%, 65%, 70%, 75%, 80%, 85%, 90% or 95% sequence identity thereto that retain the function of the human Gja10 promoter.

In one aspect the promoter comprises a sequence of contiguous nucleotides from the human Gja10 promoter, or variants thereof with at least 60%, 65%, 70%, 75%, 80%, 85%, 90% or 95% sequence identity to the full length of the human Gja10 promoter, wherein the variant retain the function of the human Gja10 promoter. In one aspect the promoter comprises a sequence of contiguous nucleotides from the human Gja10 promoter, or variants thereof with at least 60%, 65%, 70%, 75%, 80%, 85%, 90% or 95% sequence identity to 400 nt, 450 nt, 450 nt, 500 nt, 600 nt, 700 nt, 800 nt, 900 nt, 1.0 kb, 1.5 kb, 2.0 kb, 2.5 kb, 3.0 kb or 3.5 kb of the human Gja10 promoter.

In one aspect the promoter comprises a sequence of contiguous nucleotides from the mouse Gja10 promoter, or variants thereof that retain the function of the Gja10 promoter. In one aspect, the promoter comprises a contiguous sequence of at least 400 nt, 450 nt, 500 nt, 600 nt, 700 nt, 800 nt, 900 nt, 1.0 kb, 1.5 kb, 2.0 kb, 2.5 kb, 3.0 kb or 3.5 kb of the sequence of the mouse Gja10 promoter, which retains the function of the Gja10 promoter. In one aspect, the promoter comprises a contiguous sequence of no more than 400 nt, 450 nt, 450 nt, 500 nt, 600 nt, 700 nt, 800 nt, 900 nt, 1.0 kb, 1.5 kb, 2.0 kb, 2.5 kb, 3.0 kb or 3.5 kb of the sequence of the mouse Gja10 promoter, which retains the function of the Gja10 promoter. In one aspect the promoter comprises a sequence of contiguous nucleotides from the mouse Gja10 promoter, or variants thereof with at least 60%, 65%, 70%, 75%, 80%, 85%, 90% or 95% sequence identity thereto that retain the function of the Gja10 promoter. In one aspect the promoter comprises a sequence of contiguous nucleotides from the mouse Gja10 promoter, or variants thereof with at least 60%, 65%, 70%, 75%, 80%, 85%, 90% or 95% sequence identity to the full length of the mouse Gja10 promoter, wherein the variant retain the function of the Gja10 promoter. In one aspect the promoter comprises a sequence of contiguous nucleotides from the mouse Gja10 promoter, or variants thereof with at least 60%, 65%, 70%, 75%, 80%, 85%, 90% or 95% sequence identity to 400 nt, 450 nt, 450 nt, 500 nt, 600 nt, 700 nt, 800 nt, 900 nt, 1.0 kb, 1.5 kb, 2.0 kb, 2.5 kb, 3.0 kb or 3.5 kb of the mouse Gja10 promoter.

In one aspect, the promoter comprises the sequence of SEQ ID NO: 1, or a sequence with at least 60%, at least 70%, at least 80%, or at least 90% sequence identity thereto which retains the function of the Gja10 promoter. In another aspect, the promoter comprises a contiguous sequence of at least 400 nt, 500 nt, 600 nt, 700 nt, 800 nt, 900 nt, 1.0 kb, 1.5 kb, 2.0 kb, 2.5 kb, 3.0 kb or 3.5 kb of the sequence of SEQ ID NO: 1 which retains the function of the Gja10 promoter. In one aspect, the promoter comprises a sequence with at least 60%, at least 70%, at least 80%, or at least 90% sequence identity to the full length of SEQ ID NO: 1, wherein the variant retain the function of the Gja10 promoter. In one aspect, the promoter comprises a sequence with at least 60%, at least 70%, at least 80%, or at least 90% sequence identity to 400 nt, 500 nt, 600 nt, 700 nt, 800 nt, 900 nt, 1.0 kb, 1.5 kb, 2.0 kb, 2.5 kb, 3.0 kb or 3.5 kb of sequence of SEQ ID NO: 1.

One or more other regulatory elements may also be present as well as the promoter. For example, the promoter of the invention can be used in tandem with one or more further promoters or enhancers or locus control regions (LCRs).

A nucleic acid molecule of the invention may be administered by a gene therapy vector, liposome, nanoparticle (for example, a polymeric nanoparticle, solid lipid nanoparticle, or compacted DNA nanoparticle), a dendrimer, polyplex, or polymeric micelles.

Vectors

When the nucleic acid molecule of the invention is administered by a gene therapy vector, the vector may be of any type. For example, the vector may be a plasmid vector or a minicircle DNA vector.

Typically, vectors of the invention are viral vectors. The viral vector may be based on the herpes simplex virus, adenovirus or lentivirus. Adeno-associated virus (AAV) vectors or derivatives thereof are particularly attractive as they are generally non-pathogenic; the majority of people have been infected with this virus without adverse effects. Furthermore, the immune privilege of ocular tissue renders the eye largely exempt from the adverse immunological responses.

Vectors of the invention typically comprise two inverted terminal repeats (ITRs), preferably one at each end of the genome. An ITR sequence acts in cis to provide a functional origin of replication and allows for the integration and excision of the vector from the genome of a cell. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2, and VP3 or variants thereof. These proteins make up the capsid of an AAV viral particle.

The AAV genome may be from any naturally derived serotype or isolate or clade of AAV.

The skilled person can select an appropriate serotype, clade, clone or isolate of AAV for use in the present invention based on their common general knowledge. In a preferred aspect, the serotype is AAV8. Other serotypes may be preferred when targeting cells other than horizontal cells. For example, AAV2/5 or AAV2.

Preferably the AAV genome will be derivatized for administration to patients. Typically, it is possible to truncate the AAV genome significantly to include minimal viral sequence yet retain the above function. Derivatisation reduces the risk of recombination of the vector with the wild-type virus, and avoids triggering a cellular immune response by the presence of viral gene proteins in the target cell. A derivative may be a chimeric, shuffled or capsid-modified derivative of one or more naturally occurring AAV viruses.

The genome of all AAV serotypes can be enclosed in a number of different capsid proteins. AAV2, for example, can be packaged in its natural AAV2 capsid (AAV2/2) or it can be pseudotyped with other capsids (e.g. AAV2 genome in AAV1 capsid; AAV2/1, AAV2 genome in AAV5 capsid; AAV2/5 and AAV2 genome in AAV8 capsid; AAV2/8).

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2.

Increased efficiency of gene delivery may be affected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins may be generated by engineering the capsid protein sequences to transfer specific capsid protein domains, surface loops or amino acid residues between different capsid proteins. Shuffled capsid proteins may be generated by DNA shuffling or by error-prone PCR.

The sequences of capsid genes may also be genetically modified to introduce specific deletions, substitutions or insertions to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one that assists purification of the viral particle as part of the production process i.e. an epitope or affinity tag. The site of insertion will typically be selected so as not to interfere with other functions of the viral particle e.g. internalization, trafficking of the viral particle. The skilled person can identify suitable sites for insertion based on their common general knowledge.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

AAV viruses are replication-incompetent. Therefore helper virus functions, preferably adenovirus helper functions, will typically also be provided on one or more additional constructs to allow for AAV replication.

For the avoidance of doubt, the invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The invention additionally provides a host cell comprising a vector or AAV viral particle of the invention.

The vector of the invention may be prepared by standard means known in the art for the provision of vectors for gene therapy. Thus, well established public domain transfection, packaging and purification methods can be used to prepare a suitable vector preparation.

A particularly preferred packaged viral vector for use in the invention comprises an AAV8 serotype or a derivatized genome of AAV2 in combination with AAV5 or AAV8 capsid proteins.

All of the above additional constructs may be provided as plasmids or other episomal elements in the host cell. Alternatively, one or more constructs may be integrated into the genome of the host cell.

Exogenous Proteins

Improved contrast sensitivity may result from the delivery of exogenous components that cause hyperpolarization of horizontal cells. For example, one or more of ion channels, ion pumps, or proton pumps that conduct net outward currents leading to hyperpolarization or receptors linked to ion channels or ion pumps, wherein activation or inactivation by endogenous or exogenous molecules leads to hyperpolarization of horizontal cells.

Means of delivering protein therapeutics to cells are known in the art. For example, proteins of the invention may be conjugated to a peptide which aid delivery to the horizontal cells. Peptides of the invention, such as h4mDi, may be conjugated to a cell-penetrating peptide (CPPs). CPPs of the invention aid the uptake of substances into tissues. CPPs of the invention aid the intracellular update of substances. CPPs of the invention enhance the transcytosis of substances. Intracellular update can be measured by the skilled person by methods known in the art.

Small Molecules

Also provided are small molecules that may be administered to horizontal cells leading to their hyperpolarization. For example small molecules that block ion channels or ion pumps that constitutively conduct net inward currents or that block receptors or signalling components up or downstream of such conductances would help to bring about horizontal cell hyperpolarization. Small molecules may be delivered to horizontal cells using liposomes, CPPs, intravitreal injection or combination thereof.

Pharmaceutical Compositions

The invention provides a pharmaceutical composition comprising a nucleic acid (coding sequence, expression construct, or vector), protein or small molecule of the invention. The composition may additionally comprise a pharmaceutically acceptable carrier, diluent, excipient, adjuvant, buffer, stabilizer, and/or other materials well known to those skilled in the art. Such materials should be non-toxic and should not interfere with the efficacy of the active ingredient.

The precise nature of the carrier or other material may be determined by the skilled person according to the route of administration; for example, direct retinal, subretinal or intravitreal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Hartmann's solution. Preservatives, stabilizers, buffers, antioxidants and/or other additives may be included, as required.

For delayed-release, the vector may be included in a pharmaceutical composition that is formulated for slow release, such as in microcapsules formed from biocompatible polymers or in liposomal carrier systems according to methods known in the art. Dosages and dosage regimes can be determined within the normal skill of the medical practitioner responsible for the administration of the composition. The dosage of the active agent(s) may vary, depending on the reason for use, the individual subject, and the mode of administration. The dosage may be adjusted based on the subject's weight, the age and health of the subject, and tolerance for the compound(s) or composition.

Host Cells

The invention additionally provides a host cell comprising a nucleic acid molecule (coding sequence, expression construct, or vector), or AAV viral particle disclosed herein. Any suitable host cell can be used to produce the nucleic acid molecule (for example, a coding sequence, expression construct, or vector) disclosed herein. In general, such cells will be transfected mammalian cells, but other cell types, e.g. insect cells, can also be used. In terms of mammalian cell production systems, HEK293 and HEK293T are preferred for AAV vectors. BHK or CHO cells may also be used.

Methods of Therapy and Medical Uses

Nucleic acid molecules (including coding sequences, expression constructs and vectors), proteins, small molecules and compositions of the invention can be used in a method for treating and/or preventing retinal disorders.

In particular, the disclosed nucleic acid molecules and compositions may be used in a method for treating retinal disorders resulting from the loss of cone photoreceptors or loss of cone photoreceptor function. Exemplary disorders include inherited retinal disorders or acquired retinal disorders. For example, the disclosed nucleic acid molecules and compositions may be used in a method of treating any one of achromatopsia, blue cone monochromatism (BCM), macular degeneration (including age related macular degeneration and Stargardt disease) and cone-rod dystrophies.

For example, provided herein are nucleic acid molecules (including coding sequences, expression constructs and vectors) for use in a method for treating and/or preventing retinal disorders, wherein the nucleic acid molecules comprise a promoter operably linked to a nucleic acid molecule encoding an optogenetic actuator comprising a potassium channel. Also provided herein are nucleic acid molecules for use in a method for treating and/or preventing retinal disorders, wherein the nucleic acid molecules comprise a promoter operably linked to a nucleic acid molecule encoding an optogenetic actuator comprising a light-sensitive potassium channel. Also provided herein are nucleic acid molecules for use in a method for treating and/or preventing retinal disorders, wherein the nucleic acid molecules comprise a promoter operably linked to a nucleic acid molecule encoding an optogenetic actuator comprising a light-controlled membrane protein linked to potassium channel.

In a preferred embodiment, the optogenetic actuator is selected from BLINK2, BLINK1, or a photoactivated adenylyl cyclases (PACs) linked to a small cyclic nucleotide-gated potassium channel, such as NgPAC1, bPAC, TpPAC, OaPAC and lc-PAC. Also provided is an expression construct comprising a promoter operably linked to nucleic acid molecule encoding a variant of one of the aforementioned optogenetic actuators, wherein the variant optogenetic actuator functions to mediate potassium conductance.

Also provided is a method for treating or preventing retinal disorders in a patient in need thereof, said method comprising administering a therapeutically effective amount of the above-mentioned nucleic acid molecule or composition to the patient.

Also provided is the use of the above-mentioned nucleic acid molecule or composition in a method for treating or preventing retinal disorders or dystrophies in a patient in need thereof.

Also provided is the use of a nucleic acid molecule or composition in the manufacture of a medicament for the treatment or prevention of retinal disorders.

The disclosed nucleic acid molecules and compositions may be administered to patients with normal foveal function or patients who rely on eccentric fixation. The patient may be asymptomatic and/or may have a predisposition to the disease. As such, the invention also provides a method or use that comprises a step of identifying whether or not a subject is at risk of developing or has, one of the above-mentioned retinal disorders.

A prophylactically effective amount of the disclosed nucleic acid molecule, small molecule or composition may be administered to a subject. A prophylactically effective amount in the context of the present invention is an amount which prevents the further progression of the disease with or without noticeable improvement to remaining vision.

Alternatively, administration of the disclosed nucleic acid molecule, small molecule or composition after the symptoms of the disease have appeared in a subject may result in a noticeable improvement to remaining vision. Thus, as used herein, “therapeutically effective amount” means an amount of a nucleic acid set forth herein that, when administered to a mammal, is effective in producing the desired therapeutic effect.

Retinal disorders associated with loss of cone photoreceptor function include achromatopsia, blue cone monochromatism (BCM), macular degeneration (including age related macular degeneration and Stargardt disease) and cone-rod dystrophies. Therefore, beneficial or desired clinical results of the invention include increased lateral gain control, increased contrast sensitivity, increased acuity at a given contrast, higher flicker fusion thresholds and/or reduced adversity to light levels that are considerably lower than those tolerated by normal vision subjects or compared to the same patient prior to treatment. Gene supplementation according to the invention may preserve or restore photoreceptor function, resulting in an improvement in vision over a wider area than the one directly treated due to preserved or restored lateral input.

The nucleic acid molecule and pharmaceutical composition may be administered to the patient by direct retinal, subretinal or intravitreal injection. This includes direct delivery to horizontal cells. The nucleic acid molecules and compositions may specifically target horizontal cells without entering any other cell populations.

The dose of the nucleic acid molecule or compositions as disclosed herein may be determined according to various parameters, especially according to the age, weight, and condition of the patient to be treated, the route of administration, and the required regimen. A physician will be able to determine the required route of administration and dosage for any particular patient.

A typical single dose is between 10¹⁰ and 10¹² genome particles, depending on the amount of remaining retinal tissue that requires transduction. A genome particle is defined herein as an AAV capsid that contains a single-stranded DNA molecule that can be quantified with a sequence-specific method (such as real-time PCR). That dose may be provided as a single dose but may be repeated for the fellow eye or in cases where the nucleic acid, such as a vector, as disclosed herein may not have targeted the correct region of retina for whatever reason (such as surgical complication). The treatment is preferably a single permanent treatment for each eye, but repeat injections, for example in future years and/or with different AAV serotypes may be considered.

The nucleic acid molecule and composition disclosed herein can be used in combination with any other therapy for the treatment and/or prevention of the retinal disorders.

The nucleic acid molecule and composition disclosed herein may be packaged into a kit. The kit may include instructions for use.

### EXAMPLES

The inventors investigated the role of center-surround organization in vision by using a combination of psychophysics in subjects with normal vision and with specific inherited retinal diseases, together with retinal circuit manipulation in equivalent mouse models and theoretical modeling.

### Example 1. Methods

Psychophysics Experiments in Human Subjects

This study adhered to the tenets of the Declaration of Helsinki and was approved by the Moorfields Eye Hospital Ethics Committee. Informed consent was obtained from subjects or consent and assent were obtained from parents and children, respectively, prior to entering the study. All psychophysical tests involved a 2-alternative forced-choice task to detect the presence of stimuli on the left or right of a central fixation point. A chin/headrest maintained head position and viewing distance during the tasks. Stimuli were presented via a gamma-corrected CRT monitor, placed 0.8 m from the chin rest. All tasks were performed binocularly, except for experiments in Stargardt patients for which the left eye was patched. Stimuli were generated in MATLAB (MathWorks Inc, Natick, Mass., USA) and delivered to the CRT monitor via a Visage MKII Stimulus Generator (Cambridge Research Systems Ltd, Rochester, UK). Contrast sensitivity thresholds were determined using a QUEST algorithm procedure (48). All tests used a fixed number of reversals (minimum of 14 and a maximum of 28) to estimate a subject's threshold. Stimuli were presented for two seconds and subjects were allowed to respond without a timeout limit. Threshold values have been presented as contrast sensitivity (calculated as the inverse of the estimated threshold value). Normal vision subjects included 12 males and 10 females, aged 18-52. Three subjects completed all tests. All other subjects completed a subset of tests. Subjects had to fixate a fixation spot placed in the center of the monitor.

Stimuli were presented at the same elevation as the fixation spot and at the specified and identical left or right eccentricity. Presentation either left or right was randomized and subjects were asked to report where they perceived the stimulus by pressing a left or right button on a console. For Stargardt patients (and corresponding control experiments in normal vision subjects) the location of the stimulus presentation was chosen based on microperimetry data (see below). For the majority of experiments, the silent substitution technique (Estevez et al. 1982) was used to exclude S-cone activation. The rationale for this method was to negate possible differences arising from previously reported anatomical isolation of the S-cone pathway from interaction with rod input. All tests in patients with disrupted cone function (achromatopsia and blue cone monochromacy) were performed with non-selective stimuli modulated along a black-to-white axis. Comparative tests using black-to-white stimuli in normal vision subjects confirmed the main finding. Temporally modulated stimuli were presented as Michelson contrast sinusoids. A 0.2 s ramp was used at the beginning of the stimulus. Gabor patches were used for spatially modulated stimuli. The range of contrasts presented varied between 0.1 and 30%. No measured threshold reached these minimum or maximum values. Patients with Bornholm eye disease only express three classes of opsin in the retina (in two of the patients tested, the expression was S-/M-/Rhodopsin and in the third, S-/L-/Rhodopsin). Using a CRT monitor that enables isolation of up to three classes of opsin, the inventors were able to generate rod photoreceptor-isolating stimuli for these patients, allowing us to specifically activate rod photoreceptors with our test stimulus. Surround annuli that contained more than one luminance were calibrated to have the same area dedicated to each luminance.

For all annuli, ‘width’ was defined as the difference between the outer and inner radius of the annulus.

A typical testing session with a single subject would normally allow the measurement of 6-7 contrast sensitivity thresholds. No subject was tested for more than 1 hour in a single session. If a session consisted of testing a range of experimental values (for example a range of surround luminance values or stimulus frequency values) these were presented to the subject in pseudorandom order. Temporal contrast thresholds were measured using sinusoidally modulated stimuli at a range of cycle frequencies to build contrast sensitivity functions. Single measurement tests were performed at 4 Hz cycle frequency. Spatial contrast thresholds were measured by presenting a Gabor patch with a range of fixed spatial frequencies, at varying contrasts. Conversely, spatial acuity measurements were performed by presenting a 40% contrast Gabor patch at varying spatial frequencies. In a small set of experiments, the surround changed only during the presentation of the stimulus in the center (to test for/avoid adaptation to the mismatched surround). In this case, the surround was equiluminant until the stimulus was presented and returned to equiluminant once the choice was made and the trial ended.

Microperimetry and Fundus Autofluorescence Imaging in Stargardt Disease Patients

Microperimetry was performed monocularly using the Nidek MP-1 (Nidek Technologies, Padova, Italy). Pupils were dilated and cyclopleged using a 2.5% phenylephrine hydrochloride solution and 1% tropicamide ophthalmic solution.

Before testing, the Spectralis OCT (Heidelberg Engineering, Heidelberg, Germany) was used to obtain a single transfoveal horizontal line scan. This was imported and used by the Nidek MR-1 manufacturer's software as an aid to automatically locate the anatomic fovea to facilitate accurate foveal placement of the testing grid. Testing consisted of a 4 apostilbs (1.27 cd/m²) background, Goldmann size III stimulus presented for a duration of 200 ms, and a 4 to 2 dB full-threshold bracketing test strategy. The customized testing grid consisted of 44 testing locations as previously described (46). The grid pattern was of radial design with centrally-condensed spacing and covered the macular and Para macular regions. All tests were performed under almost dark (mesopic) light conditions. The sensitivity at each retinal location was determined by iteratively adjusting the light intensity until the dimmest visible stimulus was found. The sensitivity for each test location was determined on a scale of 0 dB to 20 dB (MR-1 scale), with higher values indicating greater sensitivity. Only the right eye data was used. Results from the microperimetry were used to estimate the functional border of the scotoma for each Stargardt patient. Test locations with 0-1 dB scores (i.e., retinal locations where only the brightest stimulus was detected or no stimulus at all was detected) were defined as scotoma and stimuli were placed laterally adjacent to these locations for psychophysical testing. Before commencing the QUEST estimation of contrast sensitivity, the inventors confirmed the suitability of the location by empirically testing the stimuli locations; patients were asked if they could see a 1 degree test stimulus (a static grey circle on a black background). If necessary, the test stimulus was moved laterally until the patient reported being able to clearly see it (location as shown in the Figures). Fixation stability was determined prior to commencing microperimetry testing by sampling at 25 Hz for 5-15 s. Fundus images were acquired by white light reflectance imaging. Short wavelength (486 nm) fundus autofluorescence was obtained using a Heidelberg Spectralis Scanning Light Ophthalmoscope.

AAV Vector Production and Testing

A 3.5 Kb sequence from the mouse Gja10 promoter was amplified by PCR from mouse genomic DNA and cloned into an AAV viral vector containing the gene for Cre recombinase. AAV2/8 particles were produced and titered as previously described (Nishiguchi et al., 2015). Subretinal injections were performed in adult wild-type and Cnga3^{-/-} mice. Tests for vector specificity were performed by subretinal injection in Ai9 (lox-STOP-lox-tdTomato, Allen Institute) mice. Two injections were performed in each eye (in the superior and inferior hemisphere respectively). 2 μl of virus (titer 1×10¹³ vg/ml) were injected. When two viruses (pAAV-Gja10-Cre and pAAV-lox-STOP-lox-Hm4D) were co-injected, equal volumes of 2×10¹³ vg/ml were mixed in a vial prior to injection. Virus was diluted following purification and to the appropriate titer in sterile PBS-MK. Control injections were performed with the same volume of PBS-MK. For each mouse, one eye was randomly assigned virus injection and the other sham injection. All mice were allowed to recover for at least 3 weeks following subretinal injections before behavioural testing. For imaging of sparsely labeled horizontal cells, the inventors made use of an existing dataset, where horizontal cells were labeled by subretinal injection of AAV2/2 vector in young adult wild-type mice. The AAV2/2-YC3.6. YC3.6 vector combines YFP and CFP and is a calcium sensor (49), but it was used only for labeling purposes in this study. Quantification of horizontal cell transduction was performed by co-staining with a Calbindin antibody (Swant, dilution 1:500).

Gain Control Model Fits

The data fits shown in FIG. 2C were obtained by fitting a third-order polynomial to each data set (stimuli presented within the same surround). To test whether the effect of surround luminance is better described by an input gain control or a by response gain control model, the fit obtained for one of these data sets (75% surround, f(x)=0.33x³−1.36x²+2.11x+0.02) was divided by the ratio between this and all other luminance values. For the input gain control model, the divisive factor was applied to the independent variable in the polynomial equation. For the response gain control model, the divisive factor was applied to the dependent variable.

Mouse Optomotor Behaviour

Mice were tested for Optomotor reflex in a setup (Cerebral Dynamics, NY, USA) consisting of 4 computer screens, simulating a rotating drum. An experimenter blind to the content of the subretinal injection performed scoring of the behaviour. A clockwise vs counter-clockwise 2 Alternative Forced Choice was used. The threshold was determined once the test completed 7 reversals within a 1% contrast. All acuity tests were performed at a fixed 100% contrast. Contrast sensitivity tests were performed at the indicated spatial frequencies. Mice were allowed to adapt to the set-up platform for 10 minutes on the first day before testing began. All tests were repeated four times for each condition for each mouse. To allow activation of the chemogenetic tool Hm4Di, mice were intraperitoneally injected with a Clozapine-N-oxide compound between 1 and 3 hours before testing took place. 24 hours were allowed to pass before the following test was performed, to allow termination of CNO-mediated activation of the exogenous receptor.

Optomotor tests with a thin stimulus were performed in the same setup (with the stimulus presented on all four screens). The stimulus was generated by a custom Matlab script and consisted of a left- or right-ward drifting sinusoidal, placed in the center of the screen. A QUEST Staircase procedure was used to determine the contrasts to be presented to the mouse. The surround (consisting of the entire screen except for the 1 degree height stimulus) was either black, grey (coinciding with the midpoint of the sinusoidal stimulus) or white. To avoid adaptation to a different light level, all screens were grey during interatrial intervals and the ‘surround’ was only presented simultaneously with the drifting stimulus.

Electroretinogram (ERG)

The animals were anesthetized with an intraperitoneal injection of a 0.007 ml/g mixture of medetomidine hydrochloride (1 mg/ml), ketamine (100 mg/ml), and water at a ratio of 5:3:42 before recording. Pupils were fully dilated using 1.0% tropicamide. Subdermal ground was inserted in the mouse left cheek. A drop of Viscotears 0.2% liquid gel (Dr. Robert Winzer Pharma/OPD Laboratories, Watford, UK) was placed between the electrode and the eye. Bandpass filter cut-off frequencies were 0.312 Hz and 1000 Hz. Flicker series were performed at 0.1 cd/m² light intensity as previously reported (Nishiguchi et al., 2015). The frequencies tested were 1, 3, 6, 9, 12, 15 Hz. Three repetitions were performed before and three after CNO injection. Mice were moved and repositioned after every repeat. Tests before and after CNO injection were performed on different days, as the induction time (1-3 hours) would not be covered by the same injection of anaesthetic.

Center-Surround Model and Surround Shift Model

A simple model of a photoreceptor cell synaptic terminal was built to capture the logic of the gain control center-surround interactions that the inventors identified in human and mouse vision. This model is a graded version of an integrate-and-fire neuron. The model terminal has an instantaneous linear relationship between light input and membrane potential, and a sigmoidal relationship between membrane potential and synaptic release (defined by a Hill equation). Additionally, the membrane potential tends to decay back to a resting membrane potential (Vr) over time.

The model is based on three equations:

Vᵢₙₜ=Vᵣ−i*w 1)

Vₜ=Vᵢₙₜ+(Vₜ₋₁−Vᵢₙₜ)*e^{-dt}/τ^{m} 2)

Release=Vₜ^{s}/(Vₜ^{s}+h^{s}) 3)

where Vᵣ is the resting membrane potential, i*w is the (hyperpolarizing) weighted light input, Vₜ is the membrane potential at time t, Vₜ₋₁ is the membrane potential at the previous time step, and τm is the decay time constant. Graded synaptic release from the terminal is then calculated from the membrane potential using a Hill equation with slope s and half-saturation point h (equation 3).

The model terminal was then presented with a 4% contrast, 5 Hz sine wave stimulus. Surround input was modeled to the terminal directly using a spatial receptive field model, where both the ‘center’ and the ‘surround’ are 2D spatial light stimulus weighted by a central 2D Gaussian function. In these simulations, constants were set as follows: w=0.5, s=15, h=5, and τm=10. In the data reported in the test, five input light levels were applied to the surround (values 1 to 5). Data for a progression of ten different light levels presented to the center are shown in FIG. 3k (values evenly spaced between 0.1 and 50). Modeling was performed in Python using the numpy library.

To model the effect of introducing a spatial shift between center and surround, as suggested by anatomy, the center and surround inputs were mimicked using Gaussian distributions. The effect of a stimulus where the luminance over the center is 1.2-fold higher than that in the surround was considered. The total light input to the surround distribution was modeled by assigning a weight to both center and surround luminance values (1.2 and 1, respectively, consistent with the stimulus described above) and measuring the area covered by the two stimuli. This simulation was performed for 50 different spatial shift values, where the light input to the center and the surround distribution were progressively shifted apart. Data in FIG. 3k shows result for surround Gaussian with sixteen different standard deviation values.

Histological Analysis

Eyes for histological analysis were taken post-mortem. Retinas were extracted and fixed in 4% PFA overnight. A histological clearing procedure (adapted from Costantini et al., 2015) was performed on whole retinas to maximize imaging quality and aid verification of expression of the fluorescent protein in Ai9 mice and mice co-injected with pAAV-Gja10-Cre and pAAV-EF1A-DIO-hM-4Di-EGFP. Images were obtained with a Leica Confocal Microscope.

Statistics

Unless otherwise stated, averaged data are represented as mean±standard deviation. The statistical tests used for each experiment are stated in the corresponding description of the figure.

### Example 2: Psychophysics in Subjects with Normal Vision

According to the standard center-surround organization hypothesis, antagonism results in a computation that enhances differences in stimulus intensity within the receptive field. Schematics typically indicate this opposition using ‘plus’ and ‘minus’ signs to demarcate the different effect on the response. A response suppression mechanism could in principle arbitrate this interaction by a division or subtraction of the responses in neighboring areas. This has led to the implementation of response gain modulation in center-surround models. However, following this logic, mimicking light input to horizontal cells (hyperpolarizing them) would suppress light signals in adjacent areas, making everything look dimmer and making it harder for these cells to signal changes—in other words it would make vision worse.

A key feature of response suppression is that it does not change the slope proportionally to the shift it causes to the function. This puts response suppression mechanisms in direct violation of very well supported properties of perception(10), summarized by Weber's law.

This flaw in the hypothetical function for surround inhibition is puzzling, because at single-neuron level, experimental results appear to show that the surround can exert a suppressive effect(3). Furthermore, these single cell results are used to explain perceptual effects such as the simultaneous contrast illusion (FIG. 15, FIG. 16), the Hermann grid, Mach Bands and numerous naturalistic scenarios where this could be useful, seeming to confirm lateral suppression at a perceptual level. The excellent match between the size and properties of single-cell receptive fields and psychophysical perceptive fields(11),(12) allowed the inventors to experimentally probe the function of center surround organization in human subjects to resolve the inadequacies of the suppressive model.

In a first set of experiments, the inventors characterized the effect of surround luminance on contrast sensitivity and established that both temporal and spatial contrast sensitivity thresholds were significantly improved by an equiluminant (27.7 cd/m²) surround over one with low luminance (0.15 cd/m²; FIG. 1b, FIG. 5, FIG. 17). From these findings, the treatment is expected to improve spatial and temporal contrast sensitivity. This would be evident in a clinical testing using standard measures, including Pelli-Robson charts, increased reading speed and/or the ability to see moving images, for example, when watching television.

A similar result was obtained when visual acuity was measured with a fixed contrast stimulus (FIG. 1c), indicating that maximum acuity is not only determined by anatomical features such as cell density(13) but can be controlled by surround input. From these experiments the inventors concluded the improvements in temporal and spatial contrast sensitivity (mentioned above) are relevant for high contrast stimuli, and therefore will improve visual acuity at a set contrast. Treatment may thus improve visual acuity, as measured clinically using Snellen charts at mesopic luminance levels.

The inventors then determined the dependence of the effect on spatial parameters of the surround, by varying annulus width, stimulus size and eccentricity (FIGS. 1d, 1e, 2a, 6 and FIG. 7). The results suggest that this effect is mediated by a spatial organization that is consistent with the size, features and anatomy(14),(15) of the classic center-surround receptive(3),(16),(17) and perceptive (11),(12) fields. Importantly, a minimum size radius (around 250 μm at a 6 degree eccentricity) was required to observe this effect (FIG. 1e), indicating this is not due to local feedback within the stimulus location, nor due to networks of gap junction connected cells.

The inventors then probed the effect of a variety of center and surround luminance values. A high luminance (64 cd/m²) annulus degraded contrast encoding similarly to the low luminance surround (FIG. 2a, FIG. 10). In fact, imposing a difference between surround and stimulus luminance reduced contrast sensitivity relative to the peak (equiluminant) condition (FIG. 2b, lower trace at y-intercept). Furthermore, changing the surround luminance to a different value shifted the peak contrast sensitivity to the corresponding equiluminant condition (FIG. 2b, upper trace at y-intercept, FIG. 11). These results suggest that the surround has an adaptive function, which adjusts peak contrast sensitivity to its own mean luminance.

### Example 3. An Input Gain Control Function for Surround Mean Luminance

Surround luminance may therefore play a different role than the suppressive function that is usually hypothesized. Neuronal response functions to stimuli can be ‘opposed’ or ‘inhibited’ in two different ways, either by scaling the response or by scaling the input. A combination of the two within the same neuron is of course possible. Previous work has highlighted the similarity between these two computations, under the collective term of ‘normalization’ (19). Instead, the inventors propose that only by considering them as fundamentally different mechanisms, with diverse consequences for computation, one can understand the key functions of visual processing.

The inventors first considered the internal logic of the suggestion that center-surround would provide a competitive/suppressive feedback and the associated true enhancement. In principle, response suppression would enhance lateral differences by dividing or subtracting the response of neighboring areas, essentially acting to silence them. This is perhaps why response suppression is hypothetically implemented in center-surround models. Due to the overlapping properties of normalization mechanisms, such models can adequately reproduce visual properties over a limited range of input values and therefore provide reasonable fits for some experimental data.

However, a key feature of response suppression is that, unlike an input gain control mechanism, it does not change the gain proportionally to the shift it causes to the function.

This puts response suppression mechanisms in direct violation of a class of models describing the properties of vision(10), such as Weber's law: in a case where the response suppression has a ‘divisive’ effect (FIG. 14, top right panel) the gain degrades at a rate faster than the shift. In terms of vision, this would mean that the relative sensitivity of vision would not remain proportional to the mean luminance in the surround as predicted by models such as Weber's law, but it would degrade as mean luminance increases. In a subtractive response suppression mechanism, the function shifts but the slope does not change (FIG. 14, bottom right panel).

Therefore, hypothesizing a lateral subtractive response suppression mechanism would mean supporting one of two scenarios: either a steep function that imposes maximum sensitivity (in the order of a few photons) at all light levels or a very shallow function that covers the entire visible range (10⁹ logs) and that, as a result, has an extremely poor sensitivity (in the order of 10⁵-10⁶ photons). Subtractive response suppression is therefore also in conflict with well-established properties of vision. As the properties of perception essentially described by models such as Weber's law are extremely well supported by experiments in visual perception(10),(44) as well as in other systems(45), the inventors propose that lateral response suppression is an incorrect interpretation of center surround interactions in the visual system

To determine which computation best describes the data the inventors compared the fit of an input versus a response division (FIG. 2d,e). The inventors found that the scaling of the function caused by surround luminance is well predicted by an input gain computation(18) and better than by a response gain computation. This is an important finding as it suggests that the surround should be re-interpreted as an adaptive rather than a suppressive mechanism. The ability to mathematically describe the data best through a division of the input rather than by a division of the response is evidence that the interpretation that the surround provides an adaptive rather than suppressive feedback is correct. It thus helps to overturn the current theory in favor of one where hyperpolarization of horizontal cells has the potential to improve visual function.

Previous work has remained vague on the key consequences of input versus response computations, grouping them under the collective term of ‘normalization’ (19). Division of the input or the response can both mediate a reduction in excitation. This explains why experiments such as Kuffler's classic receptive field mapping(3) have led to an erroneous acceptance of a response suppression model. However, the two computations are fundamentally different, in that only a divisive input gain can preserve relative sensitivity, respecting the fundamental properties of vision. As a consequence of luminance being encoded in proportional terms, the same luminance will appear different in two different contexts without this being a true enhancement of contrast.

The inventors experimentally verified this by measuring the magnitude of altered luminance perception in a simultaneous contrast template-match task (FIG. 15a. FIG. 16). This is evidence that there is no ‘true’ enhancement of contrast, i.e. that the enhancement does not alter the perceptual threshold and does therefore not enable a subject to see something that they would not be able to see otherwise. This is strongly supported by studies that measure contrast estimates in human subjects, which have failed to demonstrate the predicted enhancement and instead found a linear estimate of supra-threshold contrast differences(20). This strengthens the inventors' claim that the current theory should be overturned.

### Example 4. Probing of Center-Surround Interaction in Subjects with Inherited Retinal Diseases

Many circuits in the visual system have been described as having a center-surround organization (22),(23)(5)(6)(3). This includes the lateral interaction between cones and rods(5),(6),(23), which has been characterized as competitive in nature. Suppressive feedback through this circuit is thought to aid the light dependent separation of rod and cone signals, ensuring the most appropriate photoreceptor type will encode the signal. The proposed silencing of lateral photoreceptors conflicts with the disclosed hypothesis.

The inventors reasoned that testing subjects with functional deficits in specific photoreceptor classes (FIG. 1g) would allow them to directly resolve which hypothesis (competitive/suppressive versus adaptive) explains this center-surround interaction. As in normal vision subjects, the inventors found that an equiluminant surround improved contrast sensitivity in patients with cone-only vision(24) (FIG. 1i, fourth and fifth traces from the bottom in low luminescence surround, FIG. 9) and in patients(25) where isolation of single cone classes could be obtained by silent substitution(26) (FIG. 1j, third and fifth traces from the bottom in low luminescence surround).

Interestingly, the inventors also found a similar effect in these patients when using rod-isolating stimuli (FIG. 1i first, second and thirds traces from the bottom in low luminescence surround and FIG. 1j first and second traces from the bottom in low luminescence surround, FIG. 8)(25). This result shows that rod photoreceptors are functional in high mesopic conditions and are not suppressed by surround input, contradicting two conventional notions about rods. Finally, in patients lacking function in all L/M-cones(25) the effect of the lateral input gain mechanism was substantially reduced, suggesting L/M-cones mediate the surround-driven improvement in contrast encoding (FIG. 1h, first to eight traces from the bottom in low luminescence surround). Together, these results demonstrate that cones drive an input gain modulation of laterally displaced cones and rods. Furthermore the fact rod photoreceptor signaling is not suppressed by surround input is inconsistent with the standard model of center-surround interactions and instead affirms the inventors' theory.

This completes the characterization of the cell types that underlie the surround effect on contrast sensitivity/acuity. Together the patient data suggests that cones in the surround alter signal processing of both rods and cones in the center. This has never been shown before. It is highly suggestive that the horizontal cell might mediate these effects because of the known connectivity of this cell in the outer retinal circuitry.

### Example 5. Manipulation of Retinal Circuitry in Mice

In order to explore the circuitry underlying input gain modulation of laterally displaced cones and rods the inventors decided to take an interventional approach in mice.

They first tested whether mice showed, similarly to humans, a dependence on surround luminance when performing a contrast sensitivity task. To achieve this, they measured contrast sensitivity by means of optomotor behavior, driven by a thin, 1° height sinusoidal stimulus, surrounded by different luminance values (FIG. 3a, schematic). As for human subjects, they found that a mismatched surround luminance degraded contrast sensitivity to the stimulus (FIG. 3a, upper trace, FIG. 12). Consistent with the results obtained in human subjects, a darker and a brighter surround luminance affected encoding to a similar extent.

The inventors then performed the same test on a Cnga3^{-/-} mouse model of achromatopsia, lacking function in cone photoreceptors similar to the achromat patients(27). The inventors found a reduced sensitivity in an equiluminant surround compared to normal vision (wild-type) mice and a reduced change between the equiluminant and luminance mismatched conditions in Cnga3^{-/-} mice (FIG. 3a lower trace, FIG. 12).

The inventors also observed a large difference in contrast sensitivity between wild-type and Cnga3^{-/-} mice using a well-established(28),(29), high-throughput full screen optomotor behavior (FIG. 3b). This has been observed before. However, previously it was though that the intrinsic properties of rods render them incapable of performing above a certain limit. In light of the present data in mouse and humans the inventors concluded that lateral adaptation arising from cones ordinarily improves the encoding capabilities of rods at mesopic light levels and so at least partially accounts for poor visual function seen in achromat mice and humans.

Having confirmed these results are consistent with the observations in human subjects, the inventors aimed to directly manipulate this circuitry. One class of neurons known to mediate interactions between cones and other photoreceptors is horizontal cells (FIG. 1g). A specific subtype (H1) connects cones to rods and has been identified in several species(11),(23),(5),(6). Its function has been repeatedly investigated and consistently confirmed to propagate signals in a cone-to-rod direction in mice(23),(30),(5). Furthermore, its axonal projection is consistent with the size of classic center-surround organization(3),(31) and has directly been shown to contribute the classic surround of single retinal ganglion cells (6),(32). Previous work has shown that photoreceptors receive a depolarizing lateral input from horizontal cells leading to suggestions of a suppressive function(5, 6, 22). However the function of this circuit cannot be determined from these experiments.

To directly resolve this issue, the inventors manipulated horizontal cell function and measured the effect on retinal encoding as well as on behavior. If activation of horizontal cells has a suppressive effect on rod pathways as has been previously suggested (5), the inventors expected to see reduced contrast sensitivity. However, the hypothesis presented herein is correct, the hyperpolarization of horizontal cells in cone-defective Cnga3^{-/-} mice should mimic the cone-to-rod input and improve contrast sensitivity in daylight conditions. To test the hypothesis, the inventors expressed the chemogenetic hyperpolarizing actuator hM4Di in horizontal cells(33),(34), using a AAV2/8 vector system (FIG. 3c) carrying a horizontal cell specific Gja10 promoter(5),(35) (FIG. 3d; average transduction 29%±11%).

The inventors assessed the effect of horizontal cell hyperpolarization on voltage modulation in photoreceptors and bipolar cells by electroretinography (ERG). Flicker stimuli with a range of temporal frequencies were presented to anaesthetized Cnga3^{-/-} mice in which one eye was sham-injected with phosphate buffered saline (PBS) and the other had received the hM4Di vector. Analysis of the Fourier transform of ERG traces showed no significant difference in representation of the frequencies corresponding to the stimulus presented before and after injection of the chemogenetic actuator clozapine N-oxide (CNO) in PBS-injected eyes (FIG. 3e, left panel). Instead, ERGs obtained from hM4Di-treated eyes showed a significant increase in the power value at tested stimulus frequencies (FIG. 3e, right panel).

The inventors investigated whether this could also lead to an improvement in a behavioral measure of contrast encoding by performing optomotor testing. Before drug-mediated induction of hyperpolarization, there was no significant difference between hM4Di- and sham-injected eyes (FIG. 3f, left panel). However, 1-3 hours after intraperitoneal injection of the activator drug CNO, contrast sensitivity in the hM4Di-injected eyes showed a significant upward shift (FIG. 3f; right panel). These results directly show that H1 horizontal cell activation improves, rather than suppresses, rod function in daylight conditions. It also shows that the impairment of rod only-mediated vision can be partially reversed, by mimicking the effect cone photoreceptors would normally have on rods via horizontal cells(5),(23),(32),(36).

With these experiments the inventors establish a specific cellular target (horizontal cells) and demonstrate that hyperpolarization of the cellular target may be used to achieve a gain of function in rod photoreceptors. CNO is an approved anti-psychotic, and could plausibly be used as a long tem actuator in patients. The dose of the actuator may be titrated in individual patients in order to set the hyperpolarization to the optimum level rather than being entirely dependent on the expression levels achieved with the initial injection.

### Example 6. Anatomically Informed Modelling

The inventors built a simple model to capture the logic of the mechanism that their results described. A surround with a divisive input gain control function was able to recapitulate the results obtained both in human and mouse vision (FIG. 3g), whereby contrast changes are transmitted to downstream neurons not in absolute terms, but relative to the mean luminance in their surround (FIG. 3h). Detailed work on the mechanism of the synapse is consistent with their model(37). Although two Gaussians with different width but aligned peaks are widely assumed to explain center-surround organization(16),(17) (FIG. 3j, left panel), no experimental evidence exists to support this distribution of lateral input. Indeed, the classic ‘Mexican hat’ shape of center-surround can originate either from co-aligned Gaussians or two shifted Gaussians (FIG. 3j). Sparse labelling of horizontal cells revealed that they extend a long axon along the outer plexiform layer in mouse (FIG. 3i) as has been reported in other species, including human(38). This feature introduces a displacement between the distribution of the center and the surround inputs. This shift would prevent the center determining its own gain, allowing instead an independent sampling of the contextual luminance by the surround (FIG. 3k). This indicates that the anatomy is designed to allow autonomy in implementing the lateral feedback that tunes encoding, challenging, for at least one cell type, the standard difference of aligned Gaussians model. Consistent with this, a minimum displacement of the surround-mediated effect is also apparent at behavioural level (FIG. 1e).

### Example 7. Lateral Interactions in Macular Degeneration

The inventors reasoned that the function identified for center-surround organization might be relevant in common disorders where cone photoreceptors are lost in the central part of the retina, including age related macular degeneration and Stargardt disease(39), in which lateral input to nearby areas or to a spared fovea would be missing. As a result, these patients might have to rely on further eccentric retinal locations with poorer acuity and contrast sensitivity for daily tasks.

The inventors first tested whether altering luminance in a region adjacent to the stimulus, rather than fully surrounding it, could skew contrast sensitivity in normal-vision subjects. Indeed, a half-annulus with a 4° width led to a significant worsening of contrast sensitivity (FIG. 4a), showing that an artificial scotoma can cause a visual deficit in neighboring areas. This result shows that missing even just part of the normal lateral input, even in healthy people, is enough to see an effect in visual processing, suggesting that vision is likely to be compromised in patients that only have a local degeneration or dysfunction—i.e. macular degenerative diseases.

The inventors reasoned that this might also be true in patients with macular degeneration. They therefore mapped function across the central retina in patients with Stargardt disease, using microperimetry testing (FIG. 4b, FIG. 13). This approach guided the positioning of stimuli to test contrast sensitivity in locations proximal and distal to the atrophic area for each patient (FIG. 4c, FIG. 13).

Contrast sensitivity in Stargardt patients was poor compared to normal vision subjects and it was poorer proximal to the lesion compared with distal to it. To measure the effect of the surround, the inventors performed a specific comparison between contrast sensitivity thresholds within each location when surrounded by either an equiluminant or low luminance annulus.

For stimuli presented distal to degenerating areas, patients showed a surround-mediated gain in contrast sensitivity that was significantly larger than for proximal locations (FIG. 4d, e). The results obtained from this cohort of patients suggest that macular degeneration deprives remaining photoreceptors of surround input, resulting in poorer quality of vision.

Overall, the inventors show that remaining vision in patients with macular degeneration is not just poor due to the general degeneration of the tissue but also specifically because the lateral adaptation that would normally come from the macular is missing. This establishes that a patient population exists in which vision could be improved by the therapeutic strategy of the invention.

### EMBODIMENTS

1. An expression construct comprising a promoter operably linked to a nucleic acid molecule encoding a gene product, wherein the expression of the gene product in horizontal cells hyperpolarises horizontal cells.

2. The expression construct of 1, wherein the expression of the gene product in horizontal cells improves vision in a subject in need thereof.

3. The expression construct of 2, wherein the expression of the gene product in horizontal cells improves mesopic and/or photopic vision.

4. The expression construct of any one of 1 to 3, wherein the gene product is selected from a membrane protein that is endogenously expressed in human horizontal cells, a membrane protein that is endogenously expressed in other excitable cell types, or a chemogenetic or optogenetic actuator.

5. An expression construct comprising a promoter operably linked to a light-sensitive gene product, wherein the expression of the gene product in horizontal cells improves vision in a subject in need thereof.

6. The expression construct of 5, wherein the promoter is a Gja10 promoter or variant thereof having at least 70%, 75%, 80%, 85% or 90% sequence identity to the sequence of the human Gja10 promoter that maintains the function of the Gja10 promoter.

7. The expression construct of 5, wherein the promoter is a Gja10 promoter, fragment thereof or variant thereof having at least 90% sequence identity to the sequence of the human Gja10 promoter that maintains the function of the Gja10 promoter.

8. The expression construct of any one of 1 to 7, wherein the gene product mediates a hyperpolarizing conductance such as a potassium conductance.

9. The expression construct of 8, wherein the membrane protein is a potassium channel.

10. The expression construct of 4 or 8, wherein the gene product comprises a light-sensitive potassium-based actuator or light-sensitive synthetic potassium channel.

11. The expression construct of 10, wherein the light-sensitive potassium-based actuator comprises a light-controlled membrane protein linked to a potassium channel.

12. The expression construct of 10, wherein the gene product comprises a light-sensitive synthetic potassium channel.

13. The expression construct of 8-12, wherein the gene product is BLINK1 or BLINK2.

14. The expression construct of 10, wherein the light-sensitive potassium-based actuator comprises a two-component optical silencer system comprising a photoactivated adenylyl cyclase (PAC) and the small cyclic nucleotide-gated potassium channel SthK.

15. The expression construct of 14, wherein the PAC is selected from NgPAC1, bPAC, TpPAC, OaPAC or lc-PAC.

16. The expression construct of any one of the preceding aspects, wherein the promoter is a horizontal cell preferred or horizontal cell specific promoter.

17. The expression construct of 16, wherein the promoter is a Gja10 promoter, or a fragment or variant thereof.

18. The expression construct of 17, wherein the promoter has at least 70%, 75%, 80%, 85% or 90% sequence identity to the full length of the Gja10 promoter; optionally wherein the promoter is a human or mouse Gja10 promoter.

19. The expression construct of 17, wherein the promoter has at least 70%, 75%, 80%, 85% or 90% sequence identity to 400 nt, 450 nt, 500 nt, 600 nt, 700 nt, 800 nt, 900 nt, 1.0 kb, 1.5 kb, 2.0 kb, 2.5 kb, 3.0 kb or 3.5 kb of the full length of the Gja10 promoter; optionally wherein the promoter is a human or mouse Gja10 promoter.

### REFERENCES

1. R. B. MacLeod, E. Hering, L. M. Hurvich, D. Jamieson, Outlines of a Theory of the Light Sense. Am. J. Psychol. (2006), doi:10.2307/1420566.
2. H. K. Hartline, INHIBITION IN THE EYE OF LIMULUS. J. Gen. Physiol. (2004), doi:10.1085/jgp.39.5.651.
3. S. W. KUFFLER, Discharge patterns and functional organization of mammalian retina. J. Neurophysiol. (1953), doi:10.1152/jn.1953.16.1.37.
4. R. H. Masland, The Neuronal Organization of the Retina. Neuron (2012), doi:10.1016/j.neuron.2012.10.002.
5. T. Szikra, S. Trenholm, A. Drinnenberg, J. Jüttner, Z. Raics, K. Farrow, M. Biel, G. Awatramani, D. A. Clark, J. A. Sahel, R. A. Da Silveira, B. Roska, Rods in daylight act as relay cells for cone-driven horizontal cell-mediated surround inhibition. Nat. Neurosci. (2014), doi:10.1038/nn.3852.
6. M. Joesch, M. Meister, A neuronal circuit for colour vision based on rod-cone opponency. Nature (2016), doi:10.1038/nature17158.
7. E. I. Knudsen, M. Konishi, Center-surround organization of auditory receptive fields in the owl. Science (80-.). (1978), doi:10.1126/science.715444.
8. G. Westheimer, Spatial interaction in human cone vision. J. Physiol. (1967), doi:10.1113/jphysiol.1967.sp008198.
9. J. Xing, D. J. Heeger, Center-surround interactions in foveal and peripheral vision. Vision Res. (2000), doi:10.1016/S0042-6989(00)00152-8.
10. S. S. Stevens, On the psychophysical law. Psychol. Rev. (1957), doi:10.1037/h0046162.
11. R. Oehler, Spatial interactions in the rhesus monkey retina: a behavioural study using the Westheimer paradigm. Exp. Brain Res. (1985), doi:10.1007/BF00230900.
12. P. Neri, D. M. Levi, Receptive versus perceptive fields from the reverse-correlation viewpoint. Vision Res. (2006), doi:10.1016/j.visres.2006.02.002.
13. E. T. Rolls, A. Cowey, Topography of the retina and striate cortex and its relationship to visual acuity in rhesus monkeys and squirrel monkeys. Exp. Brain Res. (1970), doi:10.1007/BF00235053.
14. D. M. Dacey, M. R. Petersen, Dendritic field size and morphology of midget and parasol ganglion cells of the human retina. Proc. Natl. Acad. Sci. U.S.A (1992).
15. H. Kolb, K. A. Linberg, S. K. Fisher, Neurons of the human retina: A Golgi study. J. Comp. Neurol. (1992), doi:10.1002/cne.903180204.
16. R. W. Rodieck, J. Stone, Analysis of receptive fields of cat retinal ganglion cells. J. Neurophysiol. (1965).
17. C. Enroth-Cugell, J. G. Robson, The contrast sensitivity of retinal ganglion cells of the cat. J. Physiol. (1966), doi:10.1113/jphysiol.1966.sp008107.
18. S. R. Olsen, V. Bhandawat, R. I. Wilson, Divisive normalization in olfactory population codes. Neuron (2010), doi:10.1016/j.neuron.2010.04.009.
19. M. Carandini, D. J. Heeger, Normalization as a canonical neural computation. Nat. Rev. Neurosci. (2012), doi:10.1038/nrn3136.
20. A. P. Ginsburg, M. W. Cannon, M. A. Nelson, Suprathreshold processing of complex visual stimuli: Evidence for linearity in contrast perception. Science (80-.). (1980), doi:10.1126/science.7367887.
21. O. S. Packer, D. M. Dacey, Receptive field structure of H1 horizontal cells in macaque monkey retina. J. Vis. (2002), doi:10.1167/2.4.1.
22. O. S. Packer, J. Verweij, P. H. Li, J. L. Schnapf, D. M. Dacey, Blue-yellow opponency in primate S cone photoreceptors. J. Neurosci. (2010), doi:10.1523/JNEUROSCI.4738-09.2010.
23. J. Trümpler, K. Dedek, T. Schubert, L. P. D. S. Müller, M. Seeliger, P. Humphries, M. Biel, R. Weiler, Rod and cone contributions to horizontal cell light responses in the mouse retina. J. Neurosci. (2008), doi:10.1523/JNEUROSCI.1564-08.2008.
24. S. A. Kabanarou, G. E. Holder, F. W. Fitzke, A. C. Bird, A. R. Webster, Congenital stationary night blindness and a “Schubert-Bornschein” type electrophysiology in a family with dominant inheritance. Br. J. Ophthalmol. (2004), doi:10.1136/bjo.2003.033555.
25. J. Aboshiha, A. M. Dubis, J. Carroll, A. J. Hardcastle, M. Michaelides, The cone dysfunction syndromes. Br. J. Ophthalmol. (2016), doi:10.1136/bjophthalmol-2014-306505.
26. O. Estévez, H. Spekreijse, The “silent substitution” method in visual research. Vision Res. (1982), doi:10.1016/0042-6989(82)90104-3.
27. A. J., K. N., K. A., H. C., R. G., J. Aboshiha, N. Kumaran, A. Kalitzeos, C. Hogg, G. Rubin, M. Michaelides, A Quantitative and Qualitative Exploration of Photoaversion in Achromatopsia. Investig. Ophthalmol. Vis. Sci. (2017), doi:http://dx.doi.org/10.1167/iovs.17-21935.
28. R. A. Pearson, A. C. Barber, M. Rizzi, C. Hippert, T. Xue, E. L. West, Y. Duran, A. J. Smith, J. Z. Chuang, S. A. Azam, U. F. O. Luhmann, A. Benucci, C. H. Sung, J. W. Bainbridge, M. Carandini, K. W. Yau, J. C. Sowden, R. R. Ali, Restoration of vision after transplantation of photoreceptors. Nature (2012), doi:10.1038/nature10997.
29. K. M. Nishiguchi, L. S. Carvalho, M. Rizzi, K. Powell, S. M. K. Holthaus, S. A. Azam, Y. Duran, J. Ribeiro, U. F. O. Luhmann, J. W. B. Bainbridge, A. J. Smith, R. R. Ali, Gene therapy restores vision in rd1 mice after removal of a confounding mutation in Gpr179. Nat. Commun. (2015), doi:10.1038/ncomms7006.
30. R. Weiler, F. Zettler, The axon-bearing horizontal cells in the teleost retina are functional as well as structural units. Vision Res. (1979), doi:10.1016/0042-6989(79)90193-7.
31. T. E. Ogden, The morphology of retinal neurons of the owl monkey aotes. J. Comp. Neurol. (1974), doi:10.1002/cne.901530405.
32. S. Ströh, C. Puller, S. Swirski, M.-B. Hölzel, L. I. S. van der Linde, J. Segelken, K. Schultz, C. Block, H. Monyer, K. Willecke, R. Weiler, M. Greschner, U. Janssen-Bienhold, K. Dedek, Eliminating Glutamatergic Input onto Horizontal Cells Changes the Dynamic Range and Receptive Field Organization of Mouse Retinal Ganglion Cells. J. Neurosci. (2018), doi:10.1523/jneurosci.0141-17.2018.
33. H. Zhu, B. L. Roth, DREADD: A chemogenetic GPCR signaling platform. Int. J. Neuropsychopharmacol. (2015), doi:10.1093/ijnp/pyu007.
34. J. H. A. Lim, B. K. Stafford, P. L. Nguyen, B. V. Lien, C. Wang, K. Zukor, Z. He, A. D. Huberman, Neural activity promotes long-distance, target-specific regeneration of adult retinal axons. Nat. Neurosci. (2016), doi:10.1038/nn.4340.
35. S. Hombach, U. Janssen-Bienhold, G. Söhl, T. Schubert, H. Büssow, T. Ott, R. Weiler, K. Willecke, Functional expression of connexin57 in horizontal cells of the mouse retina. Eur. J. Neurosci. (2004), doi:10.1111/j.0953-816X.2004.03360.x.
36. M. VanLeeuwen, I. Fahrenfort, T. Sjoerdsma, R. Numan, M. Kamermans, Lateral Gain Control in the Outer Retina Leads to Potentiation of Center Responses of Retinal Neurons. J. Neurosci. (2009), doi:10.1523/jneurosci.5834-08.2009.
37. M. T. VANLEEUWEN, C. JOSELEVITCH, I. FAHRENFORT, M. KAMERMANS, The contribution of the outer retina to color constancy: A general model for color constancy synthesized from primate and fish data. Vis. Neurosci. (2007), doi:10.1017/s0952523807070058.
38. H. Kolb, E. Fernandez, J. Schouten, P. Ahnelt, K. A. Linberg, S. K. Fisher, Are there three types of horizontal cell in the human retina? J. Comp. Neurol. (1994), doi:10.1002/cne.903430304.
39. R. Allikmets, N. F. Shroyer, N. Singh, J. M. Seddon, R. A. Lewis, P. S. Bernstein, A. Peiffer, N. A. Zabriskie, Y. Li, A. Hutchinson, M. Dean, J. R. Lupski, M. Leppert, Mutation of the Stargardt disease gene (ABCR) in age-related macular degeneration. Science (80-.). (1997), doi:10.1126/science.277.5333.1805.
40. B. Lin, R. H. Masland, Populations of wide-field amacrine cells in the mouse retina. J. Comp. Neurol. (2006), doi:10.1002/cne.21126.
41. A. Drinnenberg, F. Franke, R. K. Morikawa, J. Jüttner, D. Hillier, P. Hantz, A. Hierlemann, R. Azeredo da Silveira, B. Roska, How Diverse Retinal Functions Arise from Feedback at the First Visual Synapse. Neuron (2018), doi:10.1016/j.neuron.2018.06.001.
42. F. S. Werblin, Adaptation in a vertebrate retina: intracellular recording in Necturus. J. Neurophysiol. (2017), doi:10.1152/jn.1971.34.2.228.
43. M. H. Turner, G. W. Schwartz, F. Rieke, Receptive field center-surround interactions mediate context-dependent spatial contrast encoding in the retina. Elife (2018), doi:10.7554/elife.38841.
44. M. J. Morgan, R. J. Watt, The Weber relation for position is not an artefact of eccentricity. Vision Res. (1989), doi:10.1016/0042-6989(89)90198-3.
45. S. Dehaene, The neural basis of the Weber-Fechner law: a logarithmic mental number line. Trends Cogn. Sci. (2003).
46. Tanna, P. et al. Cross-sectional and Longitudinal Assessment of Retinal Sensitivity in Patients with Childhood-Onset Stargardt Disease. Transl. Vis. Sci. Technol. (2018). doi:10.1167/tvst.7.6.10
47. Euler, T., Haverkamp, S., Schubert, T. et al. Retinal bipolar cells: elementary building blocks of vision. Nat Rev Neurosci 15, 507-519 (2014). https://doi.org/10.1038/nrn3783
48. Watson, A. B., Pelli, D. G. Quest: A Bayesian adaptive psychometric method. Perception & Psychophysics 33, 113-120 (1983). https://doi.org/10.3758/BF03202828
49. Nagai T, Yamada S, Tominaga T, Ichikawa M, Miyawaki A. Expanded dynamic range of fluorescent indicators for Ca(2+) by circularly permuted yellow fluorescent proteins. Proc Natl Acad Sci USA. 101(29):10554-9 (2004). https://doi.org/10.1073/pnas.0400417101
50. Costantini, I., Ghobril, J., Di Giovanna, A. et al. A versatile clearing agent for multi-modal brain imaging. Sci Rep 5, 9808 (2015). https://doi.org/10.1038/srep09808
51 Cosentino et al., Engineering of a light-gated potassium channel Science, Vol. 348, Issue 6235, pp. 707-710 (2015)
52 Alberto et al., A light-gated potassium channel for sustained neuronal inhibition Nat Methods 15(11):969-976 (2018)
53 Bernal Sierra et al., Potassium channel-based optogenetic silencing. Nat Commun 9, 4611 2018)

1. An expression construct comprising a promoter operably linked to a nucleic acid molecule encoding a gene product, wherein the expression of the gene product in horizontal cells improves vision.

2. The expression construct of claim 1, wherein the gene product is selected from a membrane protein that is endogenously expressed in human horizontal cells, a membrane protein that is endogenously expressed in other excitable cell types, or a chemogenetic or optogenetic actuator.

3. The expression construct of claim 2, wherein the membrane protein is a neuronal receptor, an ion channel or an ion pump.

4. The expression construct of claim 2, wherein expression of the gene product hyperpolarises horizontal cells.

5. The expression construct of any one of claims 2 to 4, wherein the gene product mediates potassium conductance.

6. The expression construct of claim 5, wherein the gene product that mediates potassium conductance is an optogenetic actuator.

7. The expression construct of claim 5 or 6, wherein the gene product that mediates potassium conductance is a light-sensitive potassium channel or a light-sensitive potassium-based actuator.

8. The expression construct of claim 3, wherein the gene product is a chemosensitive receptor linked to a potassium channel.

9. The expression construct of claim 8, wherein the gene product is h4mDi.

10. The expression construct of any one of the preceding claims, wherein the promoter is a horizontal cell-preferred promoter or a horizontal cell-specific promoter.

11. The expression construct of claim 10, wherein the promoter is a Gja10 promoter, fragment thereof or variant thereof that maintains the function of the Gja10 promoter.

12. A vector comprising the expression construct of any one of the preceding claims.

13. The vector of claim 12, wherein the vector is a viral vector.

14. The vector of claim 13, wherein the viral vector is an AAV vector.

15. The vector of claim 14, wherein the AAV vector is an AAV8 vector.

16. A pharmaceutical composition comprising the expression construct of any one of claims 1 to 11, or the vector of any one of claims 12 to 15.

17. The vector of any one of claims 12 to 15 or the pharmaceutical composition of claim 16, for use in a method of treating a retinal dystrophy.

18. The vector or pharmaceutical composition for use according to claim 17, wherein the retinal dystrophy is cone dystrophy or macula dystrophy, such as achromatopsia, blue cone monochromatism (BCM), macular degeneration (including age related macular degeneration and Stargardt disease) and cone-rod dystrophies.

19. The vector or pharmaceutical composition for use according to claim 18, wherein the cone dystrophy is characterized by the reduced cone photoreceptors or reduced function of cone photoreceptors.

20. The vector or pharmaceutical composition for use according to claim 18 or 19, wherein the cone dystrophy is characterized by global reduction of cone photoreceptors or local loss of cone photoreceptors.

21. The vector or pharmaceutical composition for use according to claim 20, wherein local loss of cone photoreceptors occurs in the macula.

22. The vector or pharmaceutical composition for use according to any one of claims 17 to 21, wherein the retinal dystrophy is selected from achromatopsia, blue cone monochromatism (BCM), macular degeneration (including age related macular degeneration and Stargardt disease) and cone-rod dystrophies.

23. The vector or pharmaceutical composition for use according to any one of claims 17 to 22, wherein vision is improved following administration of the vector or pharmaceutical composition.

24. The vector or pharmaceutical composition for use according to claim 23, wherein mesopic and/or photopic vision is improved following administration of the vector or pharmaceutical composition.

25. The vector or pharmaceutical composition for use according to any one of claims 17 to 24, wherein delivery of the vector to horizontal cells enables horizontal cells to be artificially hyperpolarised or increases the outward conductance of horizontal cells.

26. A method for treating a cone dystrophy or macula dystrophy, the method comprising administering the vector of any one of claims 12 to 15 or the pharmaceutical composition of claim 16 to a subject in need thereof.


SEQUENCE LISTING (1 sequence)

SEQ ID NO: 1 - DNA, 3503 bp, Mus musculus
gcaatagaaaccctaactaaaacaattctatttaaaattttcttcatatatatgtatgaaaacaggtaaggtttacataaatgtttttattaaataaaataatattttcttttataaaaaacatttattttatgtgtatgaatgttttgcttggctgtgtctgtgcaccacttccaggcttggttcacacagtggtcagaagagagtggatctcttgagagtagagttatgaatggttgtaatctactctgtgggtactgggaaccaattcaatcataaccaaaaccaaagccatcttccagtcccagtcatattttcttagaaacccgttgtccctttcttttcttttttacctgttttctacctctcctgcctcttctttctcccttctaattcttcccatatccatactcctcgacttcctttccatgctcacacacacacacacaaaatgtatgtgtgtgtgtatacatacatatatatgtatgtatgtatgtatgtatatgtatacacacatatatacacacatacatatatacacatgtatacatatataaacacacatatatatatgtgatcatgtatatactcatatatatgcttatataatcatatatctcttatacattgtatatgtgtacatttatggaattttattttattcagtgattaaaagtcttacaacaacctacagattaatttatgtgaggaataatagtgagtttccaattccttttggtttttcaggtgaaattacaatatatgtgtgtcccacacctcatatataccatgactatggttgagtttgtggcttctgagataatgcctaggtagaggtaacacttcagaaaaatattggagagatgaattttttaaaagatttgtttgtttgtttatttatttatttatttattacgcataatgttctgcctgcacttatgcctgcaagccaggagcgggcaccagacctcattacagatggttgtgagccaccatgcagttgctgggaattgaactcaggacctctggaagagtagacagtgcttttaacctctgagccaactctccagccctgaagagatgaatttaaaaattaaaaattatgccacggttgttggtttcaggatggaagctgagaaaactcatctcagagatagaagcttaaaaatgaaagctttgttttctgagtgctcagggaggtggccacttcagaatctgaactgcttcccagaaaggagatggtacatttttaaaggatgggagcacaaagcaagtgggtagctgggaggagctgggggagctgctgtgttatccagctttattttgacattacaggttaagctgggttaagcaaggaagtaccagttagagactgggatgcatccaggacacctggcttcaaggttctaattcagcttttgcagccaggtcccctcctggactctggactggaattttagaatgataagggagcaaagaagtgggcaagcagcacgtagtcaattagggtataacaggcaattggatatatattttatagtttatgtaccctggtttaggtaatagcagtttccatatttttttactggttaacagatgattaagaaaacattggagaagtgggacagaagttctttcctaggtgtaggaacttgaacttgtttgaccctgccagcaagatggggaatttgtccttgagatgggtggattcagacaactgtctccatacaatttgcccctgagatatctggactcagacaactgtttacaccagaggctgcttggctatggggaagtacccagctctcctacggcatgggaccttgaatttagtttcttcttatggttaaatatagtcttaaagcaaaatagtagtacttagaataagtttcttttgtttgttccttacaattgtctacttcaatttaattacattttaccctatgattttcatatctaaaaagccttccacatattagaaatctttaggttatcatgatggctagtcttcacacaaactagagctatctgtggggagggaaacttaattgagaaaatgcttccataagatttggctgtaaggtggttttttagtgattgatgtggggagggcccagtgcattatggatgattccattccaaggctggtggttctaggttctagaaaaaagcaggatgagcaagccatgaaaagcagaccagtaagcagcacccttccatagcctctgcatcagctcctgcctccaggttccagccctgcttgagttcctgtcctgactttttccaatgatgggttatgatctggaagtataagctgagtaagcaaaccctttccttcccaacttgctttttggtcatggtgttttcatagcagccatagaaaccctaagacagcgttttgagtgttttgatctaattgaaccttgaaaaacatagtttttccaggataaaaaacagtaacttgtgtatgacacttataccttgattatttcctagactccaagctttctagagaaaccatttatactaacttttccaagtttgcaagaattccataaagacacaagacacggtggcacagccctttaattctagcacttgggagtcagaggcaggcaactctgagtttgagacgagtctgctatacaaagtggattccaggacagttagggctgttatgcagagaaattctgtcttgaaaaaccaagaaaattttcaatgaagacctggctttccagatgtgttttagtggcactgtttttgtgactgcctcaacattaccactaacttgacctgaagcagctcccaaggatttgcctggctgatttctctttacaagtgcttcatcttacaatggtagcacgtgtggtatatgaggagatcctactttgctttccctgttgacagttatttcatgaaagactcaagggaatatattgatatcatctgtgtagagagatccagacaccatggagagactctacttgcagggggcgcaccttttcaggaaatgagacagaaaatgaaaacaccacaacagtcttctaggttaaatgctcaatgagtggtagtggaagcttagaaaaggggaaagtaggtgggggttggcctccttagagaaaagttctagagtatttgaatcttaagatcgtgctagatgtgagatgaatgggcctctcagataaatagaaaaataagcaagactgtggaatacattgtacaaatacggaaactaatgggtatcctagaatgaaagggaactgcatatggccaagaaaaggataagaagtaaactgagttagattatagggggtcagtgttttgttcagaaactaatactcctcggaaaattccagtttcttcaagtatgaagttgctgtcaggaattgtgttgacttgagccaagagtctgtgctactcatgagcatgcccctttataagtgacgtccctctgttttattagcctccttgcttctgtatgtttcagatgtgacttcctaacc